(19) United States
(12) Patent Application Publication
(10) Pub. No.: US 2023/0111643 A1
(43) Pub. Date: Apr. 13, 2023

(54) COMPOSITIONS, KITS AND METHODS FOR STYLING HAIR FIBERS

(71) Applicant: LANDA LABS (2012) LTD., Rehovot (IL)

(72) Inventors: Sagi ABRAMOVICH, Ra'anana (IL); Alexander BLUVSTEIN, Rishon LeZion (IL); Nir KOJOKARO, Rehovot (IL)

(73) Assignee: LANDA LABS (2012) LTD., Rehovot (IL)

(21) Appl. No.: 18/052,225

(22) Filed: Nov. 3, 2022

Related U.S. Application Data
(63) Continuation-in-part of application No. PCT/IB2021/053730, filed on May 4, 2021.

(30) Foreign Application Priority Data
May 4, 2020 (GB) 20065694
May 4, 2020 (GB) 20065736

(51) Int. Cl. A61K 8/34; A61K 8/06; A61Q 5/06; A45D 7/06

(52) U.S. Cl. CPC A61K 8/345; A61K 8/062; A61Q 5/06; A45D 7/06; A61K 2800/30

(57) ABSTRACT

The present disclosure relates to a method for styling mammalian hair fibers, including providing body, straightening, relaxing, curling, or applying any other desired modification to the shape of the hair. The method comprises applying a hair styling composition comprising at least one thermally-curable polyol monomer to the hair, allowing the monomers to penetrate within the hair and curing such monomers to internally form a polymer able to overcome the tendency of the hair to revert to its native shape. When curing is performed while the hair is in a desired modified shape, the resulting polymers may maintain the modified shape. Suitable compositions and kits allowing to prepare the same are also disclosed.

### RELATED APPLICATIONS

This application is a continuation-in-part of International Application No. PCT/IB2021/053730 filed on May 4, 2021, which claims Paris Convention priority from Great-Britain application No. 2006569.4 and from Great-Britain application No. 2006573.6, both filed on May 4, 2020. This application is also related to simultaneously filed international application No. PCT/IB2021/053720 titled “Compositions, Kits and Methods for Styling Hair Fibers”. The entire disclosures of all of the aforementioned applications are incorporated herein by reference for all purposes as if fully set forth herein.

### FIELD

The present disclosure relates to compositions, kits, and methods for styling keratinous fibers, such as mammalian hair.

### BACKGROUND

The mammalian (e.g., human) hair fiber is a layered structure, wherein the outermost layer is the cuticle, a thin protective layer made of keratin protein, surrounding a central hair shaft composed of a cortex and a medulla. The cuticle layer is built from scale-shaped cells, layered one over the other in an overlapping manner, similarly to shingles on a roof. The physical appearance and the shape of hair fibers are determined by a variety of interactions between the keratin chains within the fibers, the amino acid composition of the keratin being responsible for the types of possible interactions. Cysteine side chains allow for the formation of disulfide bonds, while other amino acids residues may form weaker interactions such as hydrogen bonds, hydrophobic interactions, ionic bonds, Coulombic interactions etc. The presence of such reactive groups in the fiber, their proportion along the fiber as well as their availability due to the fiber conformation, determine the occurrence of these interactions and the appearance of the fiber or of the hair constituted by a plurality of such fibers.

The disulfide covalent bonds that may form between two thiol side-chains of two adjacent cysteine residues account for the fibers' structure stability, durability and mechanical properties, and the breaking of these bonds by various procedures is the mechanism behind most contemporary methods of permanent hair styling (mainly straightening or waving).

One such procedure, termed “Japanese straightening”, involves reductive agents, e.g., mercaptans or sulfites, which selectively cleave the disulfide bonds, whereby the keratins mechanically relax, followed by re-oxidation of the free sulfhydryl groups, allowing for the recombination of the disulfide bonds at the end of the process, while the hair is at the conformation adapted to achieve the desired styling. Various styling means, such as hot iron or hair dryer, can be used to induce additional stress to permanently conform the hair to the desired conformation (whether straight or wavy).

Another procedure for permanent styling of the hair relies on even harsher reductive agents, such as strong alkaline agents at pH higher than 11.0. Under these conditions, the disulfide bonds are cleaved in a less selective manner when the alkaline agents deeply permeate into the pH-induced swelled hair, disrupting possible rearrangement of the disulfide bonds.

Other procedures, termed “keratin straightening” and “organic straightening”, and including “Brazilian straightening”, are considered semi-permanent, and involve the massive use of aldehydes, namely, formaldehyde, formaldehyde-producing agents, or glutaraldehyde, most straightening products containing 2-10% of such chemicals. Exemplary formaldehyde-producing agents, also referred to as formaldehyde-releasing agents, include glyoxylic acid and its derivatives (e.g., glyoxyloyl carbocysteine), some of them being commonly used as preservatives. These aldehyde-based or -producing agents react with the keratin in the hair-fibers, acting as cross-linkers, thus prolonging the durability of the new hair conformation and shape. Formaldehyde and glutaraldehyde are considered carcinogenic, and can cause eyes and nose irritation, as well as allergic reactions of the skin, eyes, and lungs. They are therefore considered hazardous by the Occupational Safety and Health Administration (OSHA), and hair styling products manufacturers are required to comply with a limit of 0.2 wt. % or less of these materials, some jurisdictions even requiring 0.1 wt. % or less. OSHA tested several keratin treatments and found that many of the products contained formaldehyde in the solution or emitted formaldehyde fume with heating even though the products were marketed as “formaldehyde free” or did not include formaldehyde in the list of ingredients, leaving the community in doubt concerning the claimed safety of such “non-formaldehyde” containing keratin-straightening products. Reports suggest that formaldehyde may simply be replaced by formaldehyde-producing agents in such products. While such products may to some extent penetrate the hair fibers under their cuticles, they are believed to mainly act by superficial coating, this external protective sheath underlying the smooth and shiny effect provided by this method. This is however a temporary effect, the coating depriving the hair of moisture, leading to brittleness, dryness and dullness of hair upon thinning of the protective keratin containing coat.

Some permanent or semi-permanent straightening methods require the use of dedicated shampoos to maintain their effect over time, such products being adapted to the particular chemical reaction each such treatment may rely on to affect hair shape. In addition, such methods show little flexibility if one wishes to further change a hair color, a hair style or to revert to the natural style, such steps typically requiring either conducting a new permanent treatment, further damaging the hair, or waiting for regrowth of hair.

The amino acids making up the keratin protein of hair fibers also contain side-chains capable of forming non-covalent weaker bonds, such as hydrogen bonds that may form between polar and/or charged side chains in the presence of water molecules. Such hydrogen bonds can form between the amino acids on the outer surface of the cuticle scales, as well as on the internal part of the scales or beneath them. Breaking of these hydrogen bonds upon exposure of the hair to heat (e.g., by a flat iron or a hair dryer, thus allowing removal of the water from the hair), and their reformation by drying or cooling, provides for temporary hair styling. While such methods do not involve reagents damaging to the hair, their effect is transient, due to the sensitivity of the fibers so shaped to water, including to ambient relative humidity.

The classification of hair styling methods between permanent, semi-permanent or temporary typically depends on the number of shampoos it may take for the hair to regain its native shape. Permanent methods may be sufficiently harsh to require growth of new hair fibers and whilst some non-transient styling may be voluntarily reversed, such methods may by themselves be damaging.

Thus, there is a need for hair styling methods, which reduce the need for hair-damaging and hazardous reagents, and advantageously may at the same time provide long-lasting style and shape for the hair.

### SUMMARY

The present disclosure relates to compositions, kits and methods comprising or using the same, for styling of hair fibers developed in order to overcome, inter alia, at least some of the drawbacks associated with traditional methods of hair styling. As used herein “styling” of hair includes any action modifying its shape in a visually detectable and desirable manner, it includes straightening or relaxing of hair, if wavy, curly or coiled; or conversely curling of hair, if the hair is relatively straighter than desired; hence any increase or decrease of the natural tendency of the hair fibers to curl.

Advantageously, the curable compositions and methods according to the present teachings allow for temporary or permanent hair styling without cleavage of disulfide bonds within the hair fiber or otherwise permanent alteration of its molecular structure. Hence, if the hair fibers have in their native (unmodified) shape prior to styling according to the present teachings a certain number of sulfur bonds, the fibers styled to have a modified shape will display essentially the same number of sulfur bonds. Alternatively, the innocuity of the present compositions and methods can be assessed by the modified hair fibers displaying essentially the same physico-chemical structure as native hair fibers. For example, in some embodiments the mechanical properties of the hair fibers are not compromised by the present compositions and methods, and some properties may even improve in particular embodiments. The fact that the chemical structure of the hair fibers is not adversely affected can be demonstrated, for example, by thermal analysis, wherein the modified and native shaped hair fibers, respectively treated/shaped or untreated by the present compositions and methods, may display at least one essentially similar endotherm temperature (as can be determined by various methods, e.g., DSC, DMA, TMA, and like methods of thermogravimetry). Endotherm temperatures of two materials or hair fibers can be considered essentially similar if within 4° C., 3° C., 2° C., or 1° C., from one another. In particular embodiments, the endotherm temperatures of the treated and untreated fibers serving as reference are measured by the same thermal analysis method, DSC being preferred.

In a first aspect of the invention, there is provided a method of styling mammalian hair fibers by modifying a shape of the fibers from a native shape to a desired modified shape, the method comprising:

a) applying to individual hair fibers a hair styling composition to cover the hair fibers, the hair styling composition comprising at least one water-insoluble thermally-curable polyol monomer (T-PM) and one or more curing facilitator miscible therewith, each having an average molecular weight of 10,000 g/mol or less, and water;

b) allowing the hair styling composition to remain in contact with the hair fibers for a period of time sufficient to ensure at least partial penetration of the T-PM(s) into the hair fibers; and

c) applying thermal energy to the hair fibers, so as to at least partially cure at least part of the T-PMs having penetrated within the hair fibers, said partial curing optionally occurring while the hair fibers are in the desired modified shape.

In some embodiments, the period of time in step b), wherein the hair styling composition is allowed to remain in contact with the hair fibers is at least 5 minutes.

In some embodiments, prior to step a) of applying the hair styling composition comprising the T-PMs, one or more of the following steps can be performed:

A—the at least one T-PM, and/or the at least one curing facilitator are pre-polymerized prior to mixing with a liquid carrier of the composition (e.g., water); and/or
B—the hair fibers are pre-treated by at least one of:

a) cleaning the hair fibers;

b) drying the hair fibers at a temperature and for a period of time sufficient to ensure breakage of at least part of a plurality of hydrogen bonds of the hair fibers; and

c) applying a pre-treating composition to the hair fibers.

As discussed in more details in the context of restyling and de-styling, the actual styling step of providing a modified shape to the hair fibers treated by the present methods, need not necessarily be performed concomitantly with the curing of the monomers progressively forming a polymer (e.g., a polyurethane-based polymer) able to overcome the tendency of the hair fibers to revert to their previous (e.g., unmodified/native/differently modified) shape. Once the polymer has formed within the hair fibers, their shape can be modified when desired at a later time. The treating method can be considered a method of styling regardless of the timeline for modifying the overall shape of the fibers, since mere formation of the polymer within the fiber may provide volume, also considered a styling effect regardless of the extent of detectability of the change.

In some embodiments, the thermal energy applied to at least partially cure at least part of the thermally-curable monomers having penetrated within the hair fibers is heat being conveyed to the hair fibers by conduction (e.g., direct contact with a styling iron), by convection (e.g., using a hot air blower, hair dryer), or by radiation (e.g., using a ceramic far infrared (IR) radiation hair dryer). In some embodiments, the thermally-curable monomers may additionally be curable by other forms of energy, for instance, by electromagnetic (EM) energy, in which case the method may further include a step of partially curing by application of the EM energy adapted to the EM-curability of the monomers. For illustration, the EM-energy may be UV-light if the monomers are UV-curable in addition of being heat-curable.

In some embodiments, the fibers treated by the present methods and the untreated fibers (or similar corresponding ones) display at least one endotherm temperature within 4° C., within 3° C., within 2° C., or within 1° C. from one another as measured by thermal analysis.

For conciseness, the materials that may serve for the preparation of the hair styling compositions that can be applied in the present method for styling of hair are detailed hereinafter with reference to the compositions, the desired properties of their ingredients, and their relative proportions, these features applying mutatis mutandis for the sake of the methods.

In a second aspect of the invention, there is provided a method of restyling hair fibers having a hair shape being a first modified hair shape achieved by the styling methods or with the hair styling compositions being further detailed herein, the restyling method comprising:
a) applying thermal energy to hair fibers having a first shape and containing in an inner part thereof a synthetic polymer having a softening temperature, the synthetic polymer being able to provide a shape to the hair fibers while at a temperature lower than its softening temperature, the application of energy being for a period of time sufficient to soften the synthetic polymer within the hair fibers; and
b) terminating the application of energy while the hair fibers are in a desired restyling second modified hair shape, the second modified hair shape being the same or different from the first shape.

In some embodiments, the fibers having the desired second shape display at least one endotherm temperature within 4° C., within 3° C., within 2° C., or within 1° C. from untreated fibers lacking the synthetic polymer as measured by thermal analysis.

In some embodiments, the application of thermal energy for restyling in step a) occurs for at least for 5 minutes, and at a temperature above the softening temperature of the polymer, for instance at a temperature of at least 50° C. In some embodiments, the temperature of restyling is sufficiently high to additionally decrease the amount of residual water within the hair fibers.

In a third aspect of the invention, there is provided a method of de-styling hair fibers having a modified hair shape achieved by the styling methods or with the hair styling compositions being further detailed herein. Namely, there is provided a method of de-styling hair fibers comprising in an inner part thereof a synthetic polymer having a softening temperature, the synthetic polymer being able to provide a shape to the hair fibers while at a temperature lower than its softening temperature, the de-styling method comprising:
I—applying thermal energy to hair fibers having a first shape, the application of energy being for a period of time sufficient to soften the synthetic polymer within the hair fibers, so that the hair fibers are at least for 10 minutes at at least 40° C., or preferably at at least 45° C.;
II—applying water during the application of energy to enable at least a partial reformation of hydrogen bonds freed by the softening of the synthetic polymer; and
III—terminating the application of energy and water while the hair fibers are devoid of contrived constraint, so as to allow the polymer to regain an unsoftened form while the hair fibers are in a natural unmodified shape.

In some embodiments, the fibers having the natural unmodified shape display at least one endotherm temperature within 4° C., within 3° C., within 2° C., or within 1° C. from untreated fibers lacking the synthetic polymer as measured by thermal analysis.

The ability to restyle or de-style hair previously treated by the present methods and compositions (i.e., hair fibers including in inner parts thereof polymers synthesized in situ by cross-linking of T-PMs) is advantageous and unexpected in the field, where traditional methods typically require applications of suitable compositions to further change hair shape.

As used herein, the term “treated” with regards to hair fibers, refers to fibers that were treated with the compositions or by the methods of the present invention, and conversely, the term “untreated” refers to hair fibers that were not treated with the compositions or by the methods herein disclosed. Notably, the present compositions and methods are relatively innocuous as compared to traditional durable hair styling, treated hair fibers displaying essentially the same physico-chemical structure as untreated hair fibers.

Hair treated by the present methods and compositions may display additional advantages, such as with respect to the mechanical properties of the treated hair and/or with respect to the types of hair that can be treated. For instance, while conventional styling methods are typically deleterious to mechanical properties of the hair, hair fibers treated according to the present teachings may display at least one tensile property (e.g., elastic modulus, break stress and toughness of the hair fibers) which is at least equal to the same property in the corresponding untreated fibers. Additionally, or alternatively, the present methods and compositions can be applied to hair already processed by conventional hair procedures, such as bleaching or coloring, whereas conventional styling methods may be incompatible.

In a fourth aspect of the invention, there is provided a hair styling composition for modifying a shape of mammalian hair fibers, the hair styling composition being selected from:
A—a single-phase composition, the single-phase including at least one water-insoluble thermally-curable polyol monomer (T-PM), water having a pH selected to increase a penetration of the monomer into the hair fibers and a co-solvent, the single-phase further including one or more curing facilitators miscible therewith, each having an average molecular weight of 10,000 g/mol or less; and
B—an oil-in-water emulsion, the emulsion consisting of a) an oil phase comprising at least one water-insoluble thermally-curable polyol monomer (T-PM) and one or more curing facilitators miscible therewith, each having an average molecular weight of 10,000 g/mol or less; and b) an aqueous phase having a pH selected to increase a penetration of the monomer into the hair fibers;
   the hair styling composition being further detailed herein and claimed in the appended claims.

The pH of the composition can be selected to facilitate at least partial penetration of the energy curable monomers into the hair fibers, said pH being different than the isoelectric point of the fibers being treated at which penetration, if any, would be minimal. In some embodiments, the pH of the hair styling composition is in a range of pH 1 to pH 3.5, or pH 5 to pH 11.

In some embodiments of the aforesaid aspects, the hair styling composition contains less than 0.2 wt. % of small reactive aldehydes (SRA), the SRA being selected from formaldehyde, formaldehyde-forming chemicals, glutaraldehyde, and glutaraldehyde-forming chemicals by total weight of the composition. In other embodiments, the hair styling composition contains less than 0.1 wt. %, less than 0.05 wt. %, less than 0.01 wt. %, less than 0.005 wt. %, or less than 0.001 wt. % of SRAs by total weight of the composition.

In some embodiments of the aforesaid aspects, the hair styling composition further comprises at least one curing facilitator (e.g., a cross-linker or a curing accelerator) miscible with the monomer.

In some embodiments of the aforesaid aspects, the hair styling composition further comprises at least one additive selected from a group comprising an emulsifier, a wetting agent, a thickening agent and a charge modifying agent, or any other like additive customary to hair styling compositions and methods of use.

In a fifth aspect of the invention, there is provided a kit for styling mammalian hair fibers, the kit comprising:

a first compartment containing at least one water-insoluble thermally-curable polyol monomer (T-PM); and

a second compartment containing either:

i. water at a pH selected to increase a penetration of the monomer into hair fibers; or
ii. at least one pH modifying agent;
wherein the kit further comprises at least one curing facilitator selected from: a cross-linker and a curing accelerator, wherein each of the at least one cross-linker and/or curing accelerator is placed in a same or different separate additional compartment(s); and
wherein mixing of the compartments produces a hair styling composition as a single-phase composition or an oil-in-water emulsion, as further detailed herein and claimed in the appended claims.

In some embodiments, the hair styling composition prepared from mixing of the kit compartments is ready to use, whereas in other embodiments, the hair styling composition needs be further diluted (e.g., with tap water) by the end-user prior to mixing of the compartments and/or application on the hair fibers.

The compositions, as can be reflected in the methods and the kits, may, in some embodiments, comprise two or more types of cross-linkers. The placement of each curing facilitator depends on whether it can spontaneously (e.g., at room temperature) react with any one of the components of the first or second compartments, the material being accordingly placed in a compartment where such reactions would not occur. If such reactions could occur with intended ingredients of any particular compartment, the curing facilitator may be placed in a separate third compartment to be mixed with the first and second compartments upon preparation of the hair styling composition as a single-phase composition or an oil-in-water emulsion.

Compartments of the kits (and their respective contents) are selected so as to avoid or reduce any reaction that would diminish the efficacy of the product during storage of the kit at a desirable storing temperature (e.g., not exceeding room temperature). In some embodiments, the first and/or third compartment is maintained in an inert environment, preferably under an inert gas, e.g., argon or nitrogen. For similar reasons, the compartments can be selected to be opaque to radiation or sealed against any factor detrimental to the stability of their contents.

In some embodiments, the kit further comprises at least one co-solvent, which may be contained in the first, second, or a separate additional compartment.

In some embodiments, the kit further comprises at least one additive selected from a group comprising: an emulsifier, a wetting agent, a thickening agent and a charge modifying agent. When the at least one additive is oil-miscible, it may be placed in the first compartment. When the at least one additive is water-miscible, it may be placed in the second compartment.

In a sixth aspect of the invention, there are provided mammalian hair fibers having a shape other than a native shape, the hair fibers comprising in an inner part thereof at least partially cured thermally-curable polyol monomers (T-PM), thermally-curable or cured polyurethane oligomers (T-PuO), or thermally-curable or cured polyurethane polymers (T-PuP); the T-PM, T-PuO or T-PuP corresponding to ingredients of hair styling compositions as further detailed herein and to at least partially cured versions of said ingredients.

Additional objects, features and advantages of the disclosure will be set forth in the detailed description which follows, and in part will be readily apparent to those skilled in the art from the description or recognized by practicing the disclosure as described in the written description and claims hereof, as well as the appended drawings. Various features and sub-combinations of embodiments of the disclosure may be employed without reference to other features and sub-combinations.

### BRIEF DESCRIPTION OF THE DRAWINGS

Some embodiments of the disclosure will now be described further, by way of example, with reference to the accompanying figures, where like reference numerals or characters indicate corresponding or like components. The description, together with the figures, makes apparent to a person having ordinary skill in the art how some embodiments of the disclosure may be practiced. The figures are for the purpose of illustrative discussion and no attempt is made to show structural details of an embodiment in more detail than is necessary for a fundamental understanding of the disclosure. For the sake of clarity and convenience of presentation, some objects depicted in the figures are not necessarily shown to scale.

In the Figures:

FIG. 1 is a schematic depiction of an image as can be captured by Focussed Ion Beam milling combined with Scanning Electron Microscopy (FIB-SEM), showing a cross-section of a reference untreated hair fiber;

FIG. 2 is a schematic depiction of an image as can be captured by FIB-SEM, showing a cross-section of a hair fiber treated with a hair styling composition according to one embodiment of the present invention;

FIG. 3 shows a Differential Scanning calorimetry (DSC) series of plots of thermal analysis of hair samples, including of a reference untreated hair sample, two hair samples treated by commercial methods, and one hair sample treated by a hypothetical innocuous composition according to one embodiment of the present invention;

FIG. 4 depicts a simplified schematic diagram of a hair styling method according to an embodiment of the present teachings;

FIG. 5A shows a photograph of untreated curly black hair fibers; and

FIG. 5B shows a photograph of similar curly black hair fibers as would appear if treated with a hair styling composition according to one embodiment of the present invention.

### DETAILED DESCRIPTION

The present disclosure relates to compositions for styling hair fibers, and more particularly to curable compositions comprising at least one water-insoluble thermally-curable polyol monomer (T-PM) capable of undergoing polymerization by any suitable reaction that creates a macromolecule (e.g., a polymer). As used herein, the term monomer is not meant to include only a single repeat molecule, and may include short oligomers, as long as their number of repeats yield a molecular weight not exceeding 10,000 g/mol, 5,000 g/mol, or 3,000 g/mol, as deemed suitable for the ability of the molecule to penetrate hair fibers. The hair styling compositions allow the delivery of the energy curable monomers to the inner parts of the hair fibers, together with any compound that may be required for their proper polymerization while within the fibers, such compounds being miscible with the monomers at this location. The compounds miscible with the monomers and facilitating their curing can be curing facilitators and/or co-solvents. Such compounds can be delivered in a same phase with the monomers, or in a distinct phase. Hair styling compositions according to the present teachings can thus be selected from single-phase compositions and oil-in-water emulsions, both typically having a pH adapted to facilitate penetration of the monomers. The facilitating pH may act by promoting: a) a sufficient opening of the hair scales, and/or b) a sufficient charging (e.g., as measurable by zeta potential) of the hair fibers and hair styling composition; and can be either acidic, in a range of pH 1 to pH 3.5 or pH 4, or mild acidic to mild alkaline, in a range of pH 5 to pH 8, or alkaline, in a range of pH 8 to pH 11, preferably between pH 9 and pH 11. In other words, a pH is deemed to favor penetration into the hair fibers if being in ranges other than the isoelectric point of the hair, which may slightly vary between 3.5 and 5, 4 and 5, or 3.5 and 4, depending on the hair fibers and their health status.

Methods of preparing and using the same and kits comprising such compositions and enabling such styling are also described.

The principles, uses and implementations of the teachings herein may be better understood with reference to the accompanying description and figures. Upon perusal of the description and figures present herein, one skilled in the art is able to implement the disclosure without undue effort or experimentation.

Before explaining at least one embodiment in detail, it is to be understood that the disclosure is not necessarily limited in its application to the details of construction and the arrangement of the components and/or methods set forth herein. The disclosure is capable of other embodiments or of being practiced or carried out in various ways. The phraseology and terminology employed herein are for descriptive purpose and should not be regarded as limiting. For instance, while reference is often made to head hair to illustrate the advantages of the present invention, it is clear that the present teachings would similarly apply to wigs, hair extensions, or eyelashes, to name a few alternatives. Thus, providing a durable hair style may be to hair attached to a human subject, to wigs or hair extensions, and the terminology further includes, by way of example, providing a durable eyelash shape, to eyelashes.

It is to be understood that both the foregoing general description and the following detailed description, including the materials, methods and examples, are merely exemplary of the disclosure, and are intended to provide an overview or framework to understanding the nature and character of the disclosure as it is claimed, and are not intended to be necessarily limiting.

In one aspect of the present invention, there is provided a method for styling mammalian hair fibers by modifying the shape of the fibers.

In the first step of the method of the present invention, a liquid hair styling composition is applied onto individual hair fibers, the liquid composition being a single-phase composition or an oil-in-water emulsion comprising water and: i) at least one water-insoluble thermally-curable polyol monomer (T-PM). If the hair styling composition is provided as a single-phase, a sufficient amount of a suitable co-solvent is provided to ensure the miscibility of the monomer with a water portion of the liquid, the aqueous media containing the co-solvent being further compatible for the miscibility of any other material desired for the polymerization of the monomers (e.g., optional curing facilitators) or for the form and applicability of the composition (e.g., an emulsifier, a wetting agent, a thickening agent, etc.). If the hair styling composition is provided as a bi-phasic emulsion, a co-solvent, if at all present, is provided to ensure at least the miscibility of the monomer with optional curing facilitators, the monomers being in an oil phase of the emulsion.

Before detailing particular compounds suitable for the present methods and compositions, it is stressed that beyond the above-mentioned ability of the monomers (and of any agent facilitating their polymerization) to penetrate within the hair fibers and to be miscible with one another once and/or as long as the curing is set to proceed, the materials need more generally to be compatible with the styling compositions, their method of preparation and their method of use. By “compatible” it is meant that the monomers, the curing facilitators, the co-solvents, or any other compatible ingredient of the present compositions, do not negatively affect the efficacy of any other compound, or the ability to prepare or use the final composition. Compatibility can be chemical, physical or both and may depend on relative amount. For illustration, a curing facilitator would be compatible if having functional groups adapted to cross-link between the monomers and/or suitable to otherwise accelerate the process. A co-solvent would be compatible if having a rate of volatility slow enough for the polymerization to proceed while the relevant materials are in a same phase. Materials would be compatible if not negatively affected by the pH of the composition, or a temperature they might be subjected to during the preparation of the composition or its use for hair styling. While not essential, all materials could be liquid at room temperature (circa 23° C.), to facilitate preparation and use, or if solid could be readily miscible with the liquid components of the composition. Moreover, materials liquid at room temperature are believed to provide an improved hair feel as compared to solid materials. If a material is solid at room temperature and its dissolution requires heating, its melting point should be low enough for the temperature of heating adapted to selectively enhance its dissolution, without prematurely triggering curing of heat curable monomers or otherwise affect their ability to polymerize. If necessary, a plasticizer can be included to maintain the hair styling composition, in particular the monomers and any other curable ingredients due to penetrate the hair fibers, liquid at room temperature.

Reverting to the pre-requisite of such compounds being able to penetrate within the hair fibers, typically following suitable opening of the hair scales, without wishing to be bound by any particular theory, it is believed that smaller molecules may more easily migrate into the fibers than larger ones. While the physical size of molecules may depend on additional factors (such as special conformation and “compactness”, or lack thereof), the molecular weight of a compound may assist estimating its ability to penetrate the fibers. In some embodiments, the materials due to polymerize within the hair fibers (e.g., the monomers and cross-linkers) or due to facilitate such polymerization (e.g., the co-solvents and curing accelerators) have an average molecular weight of no more than 10,000 g/mol, no more than 5,000 g/mol, no more than 3,000 g/mol, no more than 2,500 g/mol, no more than 2,000 g/mol, no more than 1,500 g/mol, or no more than 1,000 g/mol.

The molecular weight of molecules having a known chemical formula can be calculated based on the molecular mass of its constituting atoms, in which case the average molecular weight is simply the molecular weight assigned to the specific molecule. For compounds formed of unknown or diverse chemical formulae, such as polymers, the average molecular weight of the population of related molecules can be provided by the supplier of the material, or independently determined by standard methods, such as high pressure liquid chromatography (HPLC), size-exclusion chromatography, light scattering, gel permeation chromatography (GPC), or matrix-assisted laser desorption/ionization time-of-flight mass spectroscopy MALDI-TOF MS, and some of these methods are described in ASTM D4001 or ISO 16014-3. Average molecular weight can then be estimated by number or by weight, both being encompassed herein.

In one embodiment, the T-PMs suitable for the hair styling compositions of the present invention have at least two hydroxyl groups, and are selected from polyalkylene glycols (e.g., ethylene glycol, propylene glycol), glycerin, polyether polyols (e.g., polyethylene glycol, polypropylene glycol, polybutylene glycol or polytetramethylene ether glycol), amine-based polyols (e.g., polyethanol amine), polyester polyols (e.g., 2,3-bis[[(Z)-12-hydroxyoctadec-9-enoyl]oxy]propyl (Z)-12-hydroxyoctadec-9-enoate or castor oil) and silanes containing at least two hydroxyl groups (e.g., dibutoxy(dihydroxy)silane, dihydroxy(phenyl)silane or trihydroxy-propylsilane). Most of the aforesaid T-PMs, except for silanes, cannot self-polymerize, hence would require the presence of a curing-facilitator in the composition, the facilitator preferably being in a same phase as the monomer. When oil-in-water emulsions are desired, the T-PMs should be water-insoluble, so as to constitute the oil phase of the emulsion.

The T-PMs previously described and further detailed herein are typically oily in nature, i.e., substantially not miscible in water, and thus, in absence of suitable amounts of appropriate co-solvents, are present in the oil phase of an oil-in-water emulsion. In some embodiments, the residual solubility of the T-PMs (or of any material deemed water-insoluble) is of 5 wt. % or less, 4 wt. % or less, 3 wt. % or less, 2 wt. % or less, 1 wt. % or less or 0.5 wt. % or less, with respect to the weight of the aqueous environment wherein they are disposed at the pH of the liquid. Solubility can be assessed by the naked eye, the soluble composition (e.g., a single-phase composition) being typically clear (not turbid) at room temperature. This matter can alternatively be quantified by measuring the refractive index of the solution, comparing it to a calibration curve with known amounts of T-PMs in water.

While water-insoluble T-PMs according to the present teachings typically form hair styling compositions being oil in water emulsions, it is possible, in presence of suitable amounts (e.g., above 30 wt. %) of appropriate co-solvents to alternatively form a single-phase composition.

In some embodiments, in order to enhance the polymerization, the hair styling composition (e.g., single-phase or oil-in-water emulsion) adapted to the present hair styling method further comprises, in addition to the i) at least one T-PM: ii) at least one curing facilitator, selected from a cross-linker and a curing accelerator. Cross-linkers refer to compounds that actively participate in the curing process, and are integrated in the resulting polymer network, while curing accelerators may alternatively, or additionally, catalyze or activate the curing (e.g., by lowering the polymerization temperature or increasing its rate). Curing facilitators should preferably be oil miscible to be in a same phase as the oily monomers during their polymerization within the hair fibers. Yet, if curing accelerators are used after the application of a hair styling composition to the hair, the curing accelerators to be used in such a step can be water-soluble, assuming that the accelerating solution is aqueous.

In some embodiments, a same curing facilitator can act both as a cross-linker and as a curing accelerator. Regardless of the type of monomers and curing facilitators that may cross-link to form within the hair fiber a network able to constrain the fibers in a desired modified shape, the resulting polymer internally formed can also be referred to as a synthetic skeleton. This term is not meant to imply that the monomers are necessarily artificial (not naturally occurring), but that the resulting polymer is synthesized in situ, and not naturally occurring within hair fibers. Simply presented, the extraneous polymer is able to “lock” the hair fibers in the desired shape, overcoming the innate force of the fibers otherwise allowing them to have or regain their natural shape.

In some embodiments, the cross-linkers suitable for the hair styling compositions and methods of the present invention are isocyanates, wherein the isocyanate groups react with the hydroxyls of the T-PMs, forming urethane links to obtain polyurethanes.

In some embodiments, the cross-linkers have at least two reactive isocyanate groups, available for the reaction with the T-PMs, the presence of only two cross-linking functions leading to chain extension of the polymers, wherein additional reactive groups would be available for further cross-linking of the ever-growing polymeric chains, thus increasing the density of the three-dimensional network formed therewith. Additionally, or alternatively, a relatively higher cross-linking density can be obtained by using a relatively higher concentration of cross-linkers (or a higher ratio of cross-linkers to T-PMs). Polymers formed by curing of the T-PMs within the hair fibers with a relatively high cross-linking density are expected to form stronger skeletons for the hair styled therewith than counterparts having a relatively lower cross-linking density.

As readily appreciated by a person skilled in the art of polymerization facilitated by cross-linkers, such compounds are typically present in an amount corresponding at least to a stochiometric reaction between the cross-linkable groups of the monomers and the corresponding reactive groups of the cross-linkers. Such minimal amount might already provide for an excess of cross-linkers, if some of the cross-linkable groups of the monomers and growing oligomers are hindered, in particular as curing proceeds towards the formation of more complex polymers. Nevertheless, in some embodiments, and in particular when cross-linkers may react with one another in addition to their ability to react with the monomers, it might be desired to include such curing facilitators in excess of their mere stochiometric concentration.

Yet, the present Inventors have discovered that in some circumstances polymers formed with a relatively low cross-linking density can also be suitable. This is the case in particular when the hair fibers are damaged, for instance as a result of their health status or of having been subjected to a conventional procedure deleterious to the hair, such as bleaching or coloring. Damaged hair fibers can display discontinuities in their outer surfaces, allowing for more water to penetrate the hair shafts as compared to healthy hair fibers. When the hair fibers are exposed to high temperatures (e.g., during styling with a hot iron, or drying with hot air), the residual water which can be present within the hair cortex may undergo explosive evaporation, further enlarging the defects of the damaged hair fibers or forming new micro-pores accelerating future permeation of water, the proliferation of such voids with each elevated heating significantly detracting from the hair integrity, possibly leading to hair breakage.

Without wishing to be bound by theory, it is believed that polymers formed with a relatively low cross-linking density behave in a thermoplastic manner, namely can reversibly become softer and malleable upon heating, while being sufficiently rigid upon cooling and at ambient temperatures to maintain a desired style to the treated hair. It is believed that this relative “flowability” of polymers having a relatively low cross-linking density allows them, upon heating of the hair fibers, to block or seal the pores or voids that may be present or have formed, especially in damaged hair. This “sealing effect” is expected to reduce water re-entry into the hair over time, thus decreasing the likelihood and/or extent of explosive evaporation of trapped water upon subsequent heating. Such reduction of water re-entry can be desirable for both damaged and undamaged hair, and hence, compositions forming polymers having a thermoplastic behavior by having a relatively low cross-linking density may be applied to both hair status.

Hence, in some embodiments, when a polymeric network having a relatively low cross-linking density is desired, cross-linkers can be selected to have a relatively low number of cross-linking functions and/or be present in the composition at a relatively low concentration (or at a low ratio of cross-linkers to T-PMs).

Other features readily appreciated by a skilled person may promote a relatively low or conversely a relatively high cross-linking density of a polymeric network formed by curable monomers as herein taught. For instance, relatively shorter cross-linkers (e.g., having a relatively low MW) may form polymers with denser cross-linking/tighter 3D networks, than relatively longer cross-linkers (e.g., having a relatively high MW) which may form looser networks. It is stressed that no single feature of a cross-linker could alone determine if the hair styling composition prepared therewith will tend to have, or not, a relatively low cross-linking density/thermoplastic behavior once polymerized. Still, a relatively low concentration, of a relatively long cross-linker having a relatively low amount of cross-linking functions can be expected to favor the formation of a cured polymer having a relatively lower cross-linking density than one prepared using a relatively high concentration, of a relatively short cross-linker having a relatively high amount of cross-linking functions.

Suitable isocyanates are selected from linear or branched, substituted or unsubstituted alkyl isocyanates (e.g., hexamethylene diisocyanate or isophorone diisocyanate); aromatic isocyanates (e.g., toluene diisocyanate or methylene diphenyl diisocyanate); silane isocyanates (e.g., 3-(triethoxysilyl)-propyl isocyanate) and mixture thereof.

Advantageously, but not necessarily, cross-linkers may additionally serve to modify the pH of the composition, facilitating the opening of the cuticle scales of hair fibers to which compositions including them are applied, and allow the T-PMs, or part thereof, to penetrate the hair shaft.

Without wishing to be bound by any particular theory, it is believed that the T-PMs according to the present teachings are molecules sufficiently small (e.g., having a MW of 10,000 g/mol or less) to at least partially penetrate the fiber shaft where they may subsequently polymerize upon application of thermal energy. Penetration of the T-PMs into the hair fiber can be observed and monitored by microscopic methods, such as FIB-SEM. When polymerization is effected while the hair fibers are in a desired modified shape, the resulting thermally-curable or cured polyurethane oligomers (T-PuOs) and thermally-curable or cured polyurethane polymers (T-PuPs) may maintain the fiber in the modified shape or delay the ability of the fiber to regain its native (un-modified) shape. Such steps shall be described in more details in following sections.

In some embodiments, the curing accelerators suitable for the hair styling compositions comprising T-PMs, and methods of the present invention using the same, are strong bases at low concentrations; amines, such as primary amines (e.g., oleyl amine, n-phenylenediamine), secondary amines (e.g., triethylenetetramine), tertiary amines (e.g., 2,4,6-tris(dimethyl-aminomethyl) phenol, commercialized as DMP30, and N,N-dimethyl-p-toluidine (DMPT)) and aliphatic amines (e.g., aminoethylpiperazine); metal complexes (e.g., having as metal: Co, Mn, Ce, Fe, Al, Zn, Zr, Se or Cu), including for instance metal carboxylates such as acetyl acetonates or naphthenates; metal soaps such as aluminium stearate and magnesium stearate; and metal salen complexes such as N,N′-bis-(salicylidene)ethylenediamine complex with Fe or Mn.

It is noted in this context that while compounds have been for simplicity categorized according to their main role, such functions are not necessarily exclusive of others. For illustration, a curing accelerator, typically used as a curing catalyst, may bear cross-linkable groups, such that the curing accelerator could also serve as a cross-linker, being incorporated into the formed polymeric network.

In some embodiments, the T-PMs, curable by applying thermal energy as mentioned above, may be combined with other monomers or oligomers, being curable by electromagnetic (EM) energy, including infrared (IR) radiation or ultraviolet (UV) radiation, or may by themselves additionally have functions rendering them EM-curable. When the hair styling composition contains the two types of monomers (or oligomers), two types of energy should be applied, wherein first the T-PMs are cured by thermal energy, and subsequently the EM energy is applied to cure the EM-curable monomers or oligomers. Alternatively, UV-curing may occur first, followed by the thermal curing, or both thermal and EM types of energy can be applied concomitantly. In some embodiments, suitable UV-curable monomers are acrylate-based monomers (e.g., dipropylene glycol diacrylate, 1,6-hexanediol diacrylate, hydroxyethyl methacrylate, hydroxypropyl methacrylate, trimethylolpropane triacrylate, tripropylene glycol diacrylate, isobornyl acrylate, isobornyl methacrylate and acrylated glycerol derivatives and epoxy acrylates, such as epoxy methacrylate and bisphenol A-type difunctional epoxy acrylate).

UV-curing generally requires the use of photoinitiators, as curing accelerators, to generate free radicals, thus facilitating the UV-curing. For the purpose of UV-curing combined with the thermal curing described above, common photoinitiators that are generally utilized in such UV-curing processes can be used, e.g., 2,2-dimethoxy-1,2-diphenylethan or isopropyl thioxanthone.

While the compositions and methods according to the present teachings can be applied and implemented on hair fibers separated from a living subject (e.g., on a fur or on a wig), they are typically intended for application on hair of living mammalian subjects, in particular for use on human scalps. Therefore, while a number of cross-linkers, curing accelerators or other agents and additives as detailed hereinbelow may be used in compositions able to satisfactorily modify the shape of hair fibers, all such ingredients, as well as the T-PMs, shall preferably be cosmetically acceptable. Ingredients, compositions or formulations made therefrom, are deemed “cosmetically acceptable” if suitable for use in contact with keratinous fibers, in particular human hair, without undue toxicity, instability, allergic response, and the like. Some ingredients may be “cosmetically acceptable” if present at relatively low concentration according to relevant regulations.

When the intended hair styling compositions are single-phase compositions, they are achieved when the T-PMs are dissolved in a continuous aqueous phase containing a suitable co-solvent. When the intended hair styling compositions are oil-in-water emulsions, they are achieved when the T-PMs are emulsified and dispersed as oil droplets in a continuous aqueous phase, which may optionally further include a suitable co-solvent. Curing facilitators, when present, should be miscible with the monomers while in the hair fibers, regardless of the phase from which they may be delivered to the hair cortex.

In some embodiments, the aqueous phase of the curable hair styling composition has a pH suitable a) to provide adequate charging to the hair fibers and the composition including the T-PMs, b) to provide a suitable solubility of a compound in a medium (or on the contrary a lack thereof), and/or c) to provide suitable opening of the hair scales to facilitate penetration. While acidic pH (e.g., in a range of about 1-3.5) may also enable such effects, in some embodiments, the aqueous phase of the curable hair styling composition has an alkaline pH. Electing one non-neutral pH over another may depend on the chemical nature of the monomers and curing facilitators, some intrinsically contributing to an acidic or a basic pH, or being more potent at one pH over the other.

While the pH of the hair styling compositions of the present invention can be adjusted to have any desired non-neutral pH to inter alia lift the hair scales to facilitate penetration of the monomers, such mechanism does not rule out the existence of additional ways of introducing monomers within the fiber cortex. For instance, the monomers and agents required for their polymerization may additionally be polar enough to diffuse through the hair scales, whether or not sufficiently opened for direct migration between the hair environment and its cortex.

Regardless of the form of the styling composition, and without being bound by theory, it is believed that an alkaline pH contributes inter alia to the opening of the cuticle scales by charging the surface of the hair fibers (due to chargeable groups, generally present on the fibers, e.g., carboxyl groups), thus allowing a better penetration of the monomers into the hair shaft. The alkaline pH may also contribute to the charging of the hair styling composition, increasing the zeta potential difference (AO) between the hair and the composition, a resulting higher gradient between the two facilitating the migration of the composition constituents towards the hair fibers for better contact.

In some embodiments, the hair styling composition (e.g., oil-in-water emulsion) has a pH of least 7, at least 7.5, at least 8, at least 8.5, at least 9, at least 9.5, or at least 10. Typically, the pH of the composition does not exceed pH 11. In particular embodiments, the pH of the composition is between 8 and 10.5, between 9 and 10.5, or between 9.5 and 10.5.

Such an alkaline pH of a hair styling composition can be achieved by dispersing or dissolving the oil phase in which the T-PMs reside with an aqueous phase at a suitable pH (e.g., to respectively form an emulsion or a single-phase). The pH of the aqueous phase can be adjusted by using any suitable pH modifying agent at any concentration adapted to maintain the desired pH. Such agents include bases, such as ammonium hydroxide, sodium hydroxide, lithium hydroxide or potassium hydroxide. The pH modifying agents may also be amines, such as monoethanol-amine, diethanolamine, triethanolamine, dimethylethanolamine, diethyl-ethanolamine, morpholine, 2-amino-2-methyl-1-propanol, cocamide monoethanol-amine, aminomethyl propanol or oleyl amine. Alternatively, or additionally, other components of the hair styling composition, which are basic in nature, may provide or contribute to the alkaline pH of the composition (e.g., emulsion).

Conversely, an acidic pH of 4.5 or less, 4 or less, or 3 or less may also contribute to the opening of the hair scales. Typically, the pH of a hair styling composition having such acidic pH is at least 1, at least 1.5, or at least 2, and generally between 1 and 4, between 1 and 3, between 1.5 and 3.5, between 2 and 4, or between 2.5 and 3.5. Such an acidic pH may be obtained using acids as pH modifying agents, which can be selected from acetic acid, perchloric acid, and sulfuric acid, to name a few. Alternatively, or additionally, other components of the hair styling composition, which are acidic in nature, may provide or contribute to the acidic pH of the composition (e.g., emulsion).

Since, as above exemplified, a number of compounds present in the composition may contribute to any of its particular property or function, whether dedicated for that purpose as primary role or inherently contributing to achieve it, the property sought for the composition is typically monitored at equilibrium.

The single-phase compositions and the oil-in-water emulsions typically differ from one another by the relative amounts of water and co-solvents each may contain, thus each type will be separately discussed below. It should be noted that there might be overlap in the ranges of concentrations appropriate for each type of composition, as the relative amounts of water and co-solvents suitable for a particular type of composition also depends on the monomers, the curing facilitators, or any other additive, as well as their respective amounts.

In some embodiments, the concentration of water in the single-phase composition is at least 2 wt. %, at least 5 wt. %, at least 10 wt. %, at least 15 wt. %, or at least 20 wt. % by weight of the single-phase composition. In some embodiments, the concentration of the water is at most 40 wt. %, at most 35 wt. %, or at most 30 wt. % by weight of the single-phase composition. In particular embodiments, the concentration of the water is between 2 and 20 wt. %, between 2 and 15 wt. %, between 10 and 40 wt. %, between 10 and 30 wt. %, or between 15 and 40 wt. % by weight of the single-phase composition.

In some embodiments, the concentration of water in the oil-in-water emulsion is, at least 60 wt. %, at least 65 wt. %, or at least 70 wt. % by weight of the oil-in-water emulsion. In some embodiments, the concentration of the water is at most 90 wt. %, at most 87 wt. %, or at most 85 wt. % by weight of the oil-in-water emulsion. In particular embodiments, the concentration of the water is between 60 and 90 wt. %, between 60 and 87 wt. %, between 65 and 87 wt. %, or between 70 and 85 wt. % by weight of the oil-in-water emulsion.

Water may not be the sole “liquid carrier” of the present compositions, and in some embodiments, the hair styling compositions can further contain at least one co-solvent. The at least one co-solvent can be selected from C₁-C₁₀ alcohols having at least one hydroxyl group, such as methanol, ethyl alcohol, isopropyl alcohol, 2-methyl-2-propanol, sec-butyl alcohol, t-butyl alcohol, propylene glycol, 1-pentanol, 1,2-pentanediol, 2-hexanediol, benzyl alcohol or dimethyl isosorbide; water-miscible ethers such as di(propylene glycol) methyl ether, diethylene glycol monoethyl ether, dioxane, dioxolane, or 1-methoxy-2-propanol; aprotic solvents such as ketones (e.g., methyl ethyl ketone, acetone), dimethyl sulfoxide, acetonitrile, n-methyl pyrrolidone, di-methyl carbonate or dimethylformamide; esters, such as C₁₂-15 alkyl benzoate or dibutyl maleate; and mineral or vegetal oils, such as isoparaffinic fluids, olive oil, coconut oil or sunflower oil. In particular embodiments, the co-solvent is isopropyl alcohol. Without wishing to be bound by any particular theory, it is believed that an oily co-solvent (e.g., C₁₂₋₁₅ alkyl benzoate) may also contribute to the hydrophobicity of the final composition.

As readily appreciated by the skilled persons, some of these co-solvents can indifferently be mixed with the T-PMs of the oil phase, with the aqueous phase, or in parts with both, during the preparation of an emulsion, where the phases are distinct, or during the preparation of a single-phase, where the oil phase is dissolved in the aqueous-co-solvent phase. Therefore, when referring in the following to a combined concentration of the co-solvents, a number of situations are encompassed: a) a single co-solvent is used and mixed either with the T-PMs or with the aqueous phase; b) a single co-solvent is used and mixed with both the T-PMs and the aqueous phase; and c) two or more co-solvents are used mixed with at least one of the T-PMs and the aqueous phase. Without wishing to be bound by any particular theory, co-solvents are believed to improve the surface tension of the oil phase so as to facilitate penetration of the T-PMs, and/or to increase the miscibility cross-linkers, when present, within the T-PMs, and/or to increase the miscibility of the T-PMs within the aqueous phase to form a single-phase composition.

In some embodiments, the combined concentration of the co-solvents in the single-phase composition is at least 30 wt. %, at least 40 wt. %, or at least 50 wt. % by weight of the single-phase composition. The maximal amount of co-solvents may depend on the T-PMs being selected, as well as on the presence of any additional ingredients. In any event, the concentration of co-solvents is such that the composition is in the form of a single-phase composition. In some embodiments, the combined concentration of the co-solvents is at most 80 wt. %, at most 75 wt. %, or at most 70 wt. % by weight of the single-phase composition. In particular embodiments, the combined concentration of the co-solvents is between 30 and 70 wt. %, or between 35 and 65 wt. % by weight of the single-phase composition.

In some embodiments, the combined concentration of the co-solvents in the oil-in-water emulsion is at least 1 wt. %, at least 5 wt. %, at least 10 wt. %, at least 11 wt. %, at least 12 wt. %, or at least 13 wt. % by weight of the oil-in-water emulsion. The maximal amount of co-solvents may depend on the T-PMs being selected, as well as on the presence of any additional ingredients. In any event, the concentration of co-solvents is such that the composition is in the form of an emulsion. In some embodiments, the combined concentration of the co-solvents is at most 40 wt. %, at most 35 wt. %, or at most 30 wt. % by weight of the oil-in-water emulsion. In particular embodiments, the combined concentration of the co-solvents is between 1 and 40 wt. %, between 5 and 40 wt. %, between 10 and 40 wt. %, between 12 and 35 wt. %, or between 13 and 30 wt. % by weight of the oil-in-water emulsion.

The single-phase compositions and oil-in-water emulsions can be prepared by any suitable method. For instance, the present compositions can be manufactured by mixing a first blend including the T-PM(s), hence including a predominant portion of the oil phase, with a second liquid, including a predominant portion of the aqueous phase. These distinct sub-compositions, respectively forming a “T-PMs compartment” and an “aqueous compartment”, which include any desired additive, are each said to include a predominant portion of any of the two phases, as it cannot be ruled out that some of the compounds of an oil-in-water emulsion may actually partly migrate between the two phases. For instance, considering the polymerizable sub-composition, the T-PMs may be insignificantly miscible in water and/or prepared in presence of a co-solvent (or any other component of the emulsion) exhibiting some miscibility with water, which upon mixing with the predominantly aqueous sub-composition may merge in part with the aqueous phase. When upon mixing of the two phases, one dissolves in the other, a single-phase composition is obtained instead of an emulsion.

If the oil-in-water emulsion is prepared by mixing a T-PMs compartment with an aqueous compartment, each may comprise an amount of respective ingredient suitable to achieve desired concentration in the final oil-in-water emulsion, upon mixing of the two compartments in set ratios. By way of illustration, in some embodiments, the concentration of the combination of all T-PMs (if more than one) in the T-PMs compartment is at least 2 wt. %, at least 5 wt. %, at least 9 wt. %, at least 13 wt. % or at least 15 wt. % by weight of the T-PMs compartment. In some embodiments, the concentration of the T-PMs is at most 40 wt. %, at most 37 wt. %, at most 35 wt. %, at most 33 wt. %, or at most 32 wt. % by weight of the T-PMs compartment. In particular embodiments, the concentration of the T-PMs is between 2 and 40 wt. %, between 5 and 35 wt. %, between 5 and 33 wt. %, between 9 and 33 wt. %, or between 9 and 32 wt. % by weight of the T-PMs compartment.

As single-phase compositions and oil-in-water emulsions according to the present teachings can be prepared by any additional suitable method, other than by dissolving or emulsifying a mixture of a T-PMs compartment and of an aqueous compartment, the concentration of the T-PMs is alternatively provided by weight of the total/final composition (e.g., the single-phase or the emulsion).

In some embodiments, the combined concentration of the T-PMs (if more than one) in the hair styling composition (e.g., oil-in-water emulsion) is at least 0.1 wt. %, at least 0.5 wt. %, or at least 0.9 wt. % by total weight of the composition. In some embodiments, the concentration of the T-PMs is at most 5 wt. %, at most 3 wt. %, at most 2 wt. %, or at most 1.5 wt. % by weight of the hair styling composition. In particular embodiments, the concentration of the T-PMs is between 0.1 and 5 wt. %, between 0.5 and 5 wt. %, between 0.5 and 3 wt. %, between 0.9 and 2 wt. % or between 0.9 and 1.5 wt. % by weight of the hair styling composition.

In some embodiments, the T-PM is maintained in an inert atmosphere, such as under argon or nitrogen, in order to reduce or eliminate any environmental factors (e.g., oxygen) that could induce premature and undesirable polymerization.

In some embodiments, the combined concentration of the cross-linkers present in the hair styling composition (if more than one) is at most 10 wt. %, at most 5 wt. %, at most 2.5 wt. %, at most 2 wt. %, or at most 1.5 wt. % by weight of the total composition (e.g., oil-in-water emulsion). In some embodiments, the combined concentration of the cross-linkers is at least 0.001 wt. %, at least 0.005 wt. %, at least 0.01 wt. %, at least 0.05 wt. %, at least 0.1 wt. %, or at least 0.5 wt. % by weight of the total composition. In particular embodiments, the cross-linkers are present at a combined concentration between 0.001 and 10 wt. %, between 0.001 and 5 wt. %, between 0.005 and 5 wt. %, between 0.01 and 5 wt. %, between 0.05 and 5 wt. %, between 0.01 and 2.5 wt. %, between 0.05 and 2 wt. %, between 0.05 and 10 wt. %, between 0.1 and 5 wt. %, or between 0.5 and 1.5 wt. % by weight of the total composition. When considering the weight per weight ratio between the T-PM(s) and their cross-linkers, this ratio can be between 1:15 and 10:1, between 1:15 and 7.5:1, between 1:15 and 5:1, between 1:10 and 2.5:1, between, or 1:5 and 5:1.

When hair styling compositions for forming a polymeric network having a relatively low cross-linking density are desired, so as to obtain within the hair fibers a polymeric skeleton having a thermoplastic behavior, a relatively low concentration of cross-linkers can be utilized. In such cases, the cross-linkers can be present at a combined concentration between 0.001 wt. % and 0.5 wt. %, between 0.05 wt. % and 0.3 wt. %, or between 0.07 wt. % and 0.2 wt. % by weight of the total composition. When considering the weight per weight ratio between the T-PM(s) and their cross-linkers, the ratios adapted for a relatively low cross-linking density can be between 10:1 and 2.5:1, or between 7.5:1 and 2.5:1.

If the curing process involves thermal energy, the cross-linkers are preferably selected to provide curing at a temperature elevated relatively to ambient temperature and/or at a rate sufficiently slow at room temperature, to prevent or reduce spontaneous curing during storage and/or application of the hair styling composition. To be feasible for use on living subjects, the curing temperature of a suitable cross-linker need not be too high (e.g., the hair fibers being between 50° C. and 60° C.), and both the curing temperature and curing rate of the cross-linkers can be selected to provide curing under reasonable conditions.

In some embodiments, the combined concentration of the curing accelerators (if more than one) is of at most 30 wt. %, at most 25 wt. %, at most 20 wt. %, at most 15 wt. %, at most 10 wt. %, at most 9 wt. %, at most 8 wt. %, at most 7 wt. %, at most 6 wt. % or at most 5 wt. % by weight of the T-PM(s), the curing accelerators optionally being present at at least 0.01 wt. % of the T-PM(s). When considering the amount of the curing accelerators by weight of the total hair styling composition (e.g., oil-in-water emulsion), they are generally present in very low concentrations. In some embodiments, the combined concentration of the curing accelerators is of at most 5 wt. %, at most 3 wt. % or at most 2 wt. % by weight of the total hair styling composition, the curing accelerators optionally being present at at least 0.001 wt. % of the hair styling composition.

In some embodiments, the concentration of the curing facilitators (i.e., the combined concentration of cross-linkers and curing accelerators) present in the hair styling composition is between 0.001 wt. % and 15 wt. %, between 0.001 wt. % and 10 wt. %, between 0.001 wt. % and 5 wt. %, between 0.05 wt. % and 15 wt. %, between 0.1 wt. % and 13 wt. %, or between 0.5 wt. % and 12 wt. % of the total hair styling composition. In particular embodiments, the combined concentration of the curing facilitators is between 0.001 wt. % and 5 wt. %, between 0.005 wt. % and 5 wt. %, between 0.01 and 5 wt. %, between 0.05 and 5 wt. %, between 0.01 wt. % and 2.5 wt. %, between 0.05 wt. % and 2 wt. %, between 0.1 and 2.5 wt. %, or between 0.2 and 2 wt. % by weight of the hair styling composition.

In some embodiments, the single-phase composition or the oil-in-water emulsion may further contain at least one additive, adapted to enhance one or more properties of the hair styling composition. The additive can, for instance, be an emulsifier, a wetting agent, a thickening agent, or a charge modifying agent, or any other such ingredients traditionally found in hair styling compositions (e.g., fragrances).

The hair styling composition, if an oil-in-water emulsion, may further contain an emulsifier, so as to facilitate the formation of the emulsion and/or to prolong its stability. In some embodiments, the emulsifier is a non-ionic emulsifier, preferably having a hydrophile-lipophile balance (HLB) value between 2 to 20, between 7 to 18, between 10 to 18, between 12 to 18, between 12 to 17, between 12 to 16, between 12 to 15, or between 13 to 16 on a Griffin scale. Suitable emulsifiers can be water-soluble (e.g., having an HLB value between 8 and 20), such as polysorbates (often commercialized as Tweens), ester derivatives of sorbitan (often commercialized as Spans), acrylic copolymers (e.g., commercially available as Synthalen® W2000), and combinations thereof, or oil-soluble, such as lecithin and oleic acid (e.g., having an HLB value between 2 and 8). It is to be noted, that some constituents of the hair styling compositions selected for other functions may also serve as emulsifiers.

In order to facilitate a penetration of the T-PMs into the hair fibers, the composition should be able to properly spread over the fibers to permit adequate contact. Adequate coating of the fibers by the composition during its application is expected to favor penetration, believed to be by capillary effect, of the monomers into the hair to form the synthetic polymer able to constrain the desired shape. Proper wetting of a surface can theoretically be improved by tuning the surface tension of the hair styling composition, measured in milliNewton per meter (mN/m), to be lower than the surface energy of the fibers. Such properties can be determined by standard methods, and for instance according to procedures described in ASTM D1331-14, Method C.

Virgin hair fibers, which have not been previously treated by any kind of hair modifying treatment, typically have a surface energy of about 25-28 mN/m, whereas damaged hairs generally have a higher surface energy, chemically bleached hair fibers, for instance, being in the range of 31-47 mN/m. Among the many differences between damaged and undamaged hairs, the increased presence of naturally occurring fatty acids on undamaged hairs is believed to contribute to their relatively lower surface energy. In view of the above ranges, it can be assumed that when working with a composition having a surface tension of less than 25 mN/m, suitable wetting would be observed on all hair types. It was surprisingly found that hair styling compositions having a surface tension that is too low do not provide the expected results as far as monomer penetration is concerned. The Inventors have discovered that, counterintuitively, compositions having a surface tension relatively higher than deemed theoretically appropriate are more suitable for the purpose of the present invention. Without wishing to be bound by theory, the absence of fatty acids within the hair shaft is believed to increase the surface energy perceived within the hair to be sufficiently higher than that measurable on the outer surface of the hair to require selection of a particular range of surface tensions for compositions intended to penetrate the hair shaft.

In some embodiments, the compositions of the present invention have a surface tension between 25 and 60 mN/m, between 25 and 55 mN/m, between 25 and 50 mN/m, between 25 and 45 mN/m, between 25 and 40 mN/m, between 25 and 35 mN/m, or between 30 and 40 mN/m.

The compositions of the present invention which are suitable for virgin hair, are also appropriate for hair fibers previously treated by any conventional method that may have adversely affect their integrity or properties. However, in some embodiments, the styling compositions may display a surface tension adapted to sufficiently coat damaged hairs, while not being satisfactory enough for virgin hair fibers.

Wetting agents can be added to the composition, at any suitable concentration allowing to decrease its surface tension to be within any of the afore-described suitable ranges. Exemplary wetting agents can be silicone-based, fluorine-based, carbon-based or amine-alcohols. Silicone-based wetting agents can be silicone acrylates (such as SIU 100 by Miwon Specialty Chemical). Fluorinated wetting agents can be perfluorosulfonic acids (such as perfluorooctanesulfonic acid) or perfluorocarboxylic acids (such as the perfluorooctanoic acid). Carbon-based wetting agents can be ethoxylated amines and/or fatty acid amide (e.g., cocamide diethanolamine), fatty alcohol ethoxylates (e.g., octaethylene glycol monododecyl ether), fatty acid esters of sorbitol (e.g., sorbitan monolaurate), polysorbates and alkyl polyglucosides (e.g., lauryl glucoside). Amine-functionalized silicones can also be used as wetting agents (such as amo-dimethicone or bis-aminopropyl dimethicone), as well as alkanolamines (such as 2-amino-1-butanol and 2-amino-2-methyl-1-propanol). Wetting agents, if added, are typically present in the hair styling composition (e.g., oil-in-water emulsion) at a concentration of at least 0.001 wt. %, at least 0.01 wt. % or at least 0.1 wt. %; at most 1.5 wt. %, at most 1.4 wt. % or at most 1.3 wt. %; and optionally between 0.001 and 1.5 wt. %, between 0.01 and 1.4 wt. % or between 0.1 and 1.3 wt. % by weight of the composition.

Alternatively or additionally, some of the components of the hair styling composition present therein to serve a different function may contribute to the surface tension of the hair styling composition. The surface tension of the hair styling composition may therefore be controlled by selecting and adjusting the relative concentrations of such compounds. Co-solvents may also contribute to the wetting ability of the composition towards hair fibers, in addition to contributing by their chemical formula and relative concentration the type of hair styling composition that may be formed.

In some embodiments, a thickening agent can be added to provide a desired viscosity, generally to the aqueous phase of the oil-in-water emulsion or aqueous compartment. The viscosity should be sufficiently low to allow easy application of the composition to the hair so as to satisfactorily coat all individual fibers, but high enough to remain on the hair fibers for sufficient time and prevent dripping. A relatively low viscosity may also facilitate penetration of the T-PMs into the hairs by diffusion and/or capillarity. Exemplary thickening agents can be hyaluronic acid, poly(acrylamide-co-diallyl-dimethyl-ammonium chloride) copolymer (Poly-quaternium 7, e.g., by Dow Chemicals), quaternized hydroxyethyl cellulose (Poly-quaternium 10, e.g., by Dow Chemicals), hydroxypropyl methylcellulose, etc. Thickening agents, if added, are typically at a concentration of at least 0.1 wt. %; at most 10 wt. %; and optionally between 0.5 wt. % and 5 wt. % by weight of the aqueous phase or single-phase.

In order to facilitate the migration and/or retention of the T-PMs to the surface of the hair fibers, which in turn may increase their permeation therein, there should preferably be a difference between the zeta potential of the composition and the hair. For example, the zeta potential of the hair styling composition at its pH (or ζ_{c}) should preferably be more negative or more positive than a zeta potential of the mammalian hair fibers (or ζₕ) at the same pH. In some cases, the ingredients used in the composition may provide, in addition to any other function, sufficient charging of the composition to achieve such a gradient of zeta potential values. For instance, pH modifying agents, wetting agents and/or amine-based cross-linkers may contribute to suitable charging of the oil-in water emulsion. In some embodiments, an agent dedicated to this effect, referred to as a charge modifying agent, can be added to the composition. For illustration, a water-insoluble, non-reactive amino-silicone oils may be added to the oil phase of the emulsion to modulate its zeta potential.

In some embodiments, the difference between the zeta potential of the composition ζ_{c} and the zeta potential of the hair fibers ζₕ to be treated therewith, also termed the zeta differential or delta zeta potential (Δζ_{|c-h|}) is in absolute terms at least 5 mV, at least 10 mV, at least 15 mV, at least 20 mV, at least 25 mV, at least 30 mV, or at least 40 mV. In some embodiments, Δζ_{|c-h|} absolute value is within a range of 5 to 80 mV, 10 to 80 mV, 10 to 70 mV, 10 to 60 mV, 15 to 80 mV, 15 to 70 mV, 15 to 60 mV, 20 to 80 mV, 20 to 70 mV, 20 to 60 mV, 25 to 80 mV, 25 to 70 mV, 25 to 60 mV, 30 to 80 mV, 30 to 70 mV, 30 to 60 mV, 35 to 80 mV, 35 to 70 mV, or 35 to 60 mV. Such values are preferable to set an initial charge gradient driving inter alia the T-PM(s) (e.g., as droplets) towards the hair fibers for their penetration therein. Understandingly, such gradient decreases over time, as the materials of the compositions initially accumulates on the hair outer surface modifying its zeta potential. This process is self-terminating, the migration from the composition to the hair ceasing once the gradient becomes too low (e.g., when the delta zeta potential becomes lower than 5 mV). Zeta potential can be determined by standard methods using any equipment suitable for the measurement of charge of dispersed particles.

The composition may also comprise any other additive customary to cosmetic compositions, such as preservatives, antioxidants, bactericides, fungicides, chelating agents, vitamins and fragrances, or customary to hair styling compositions, such as hair detangling agents and hair conditioning agents, the nature and concentration of which need not be further detailed herein.

The composition may also comprise any other additive customary to the form in which the hair styling composition is to be applied, such as propellants if the composition is to be sprayed, the nature and concentration of which need not be further detailed herein.

The mixing and/or emulsification of the aforesaid materials can be performed by any method known in the art. While manual shaking may suffice, various equipment, such as a vortex, an overhead stirrer, a magnetic stirrer, an ultrasonic disperser, a high shear homogenizer, a sonicator and a planetary centrifugal mill, to name a few, can be used, typically providing more uniform compositions, for instance more homogenous populations of oil droplets in the aqueous phase of an oil-in-water emulsion.

In some embodiments, the hair styling composition can be prepared by mixing or emulsifying the contents of a T-PMs compartment and an aqueous compartment, this combination being performed soon after each of the respective parts are ready. However, in alternative embodiments, the mixing of the two compartments can be deferred. In particular when the composition comprises T-PM(s) and at least one curing facilitator (e.g., a cross-linker) prone to separate into distinct phases in a complete final composition, it may be desired to allow pre-polymerization of such materials in a same polymerizable compartment. In some embodiments, the pre-polymerization step is performed on a sole mixture of T-PM(s) and curing facilitators, and not on the entire contents of a T-PM compartment if due to include additional materials that may adversely affect the process or simply delay it. In other embodiments, self pre-polymerization is performed on the T-PM(s) alone, prior to its combination with the curing facilitators or any other component of the T-PM compartment. Such pre-polymerization can be referred to as “self pre-polymerization” and is typically available to silane-based T-PMs, the curing proceeding under suitable conditions.

Such pre-polymerization, if needed and whether or not in presence of curing facilitators, should have a long enough duration to prevent the separation of the monomers and the curing facilitators into distinct phases upon mixing with additives of the T-PM compartment and/or with the contents of an aqueous compartment to an extent significantly delaying polymerization within the hair fibers following application of the mixed composition. But the pre-polymerization should be short enough so that the oligomers that may form in this process (whether of cross-linkers or monomers by themselves or of cross-linkers and monomers ones with the others) remain sufficiently small to penetrate within the hair fibers following application of the composition. It is believed that the pre-polymerization results in the formation of oligomers (regardless of composition) at the expense of the relevant building blocks (e.g., monomers and/or cross-linkers) present in the pre-polymerized compartment. This process can be monitored by a viscosity of the pre-polymerized mixture of monomers and curing facilitators increasing with time. The pre-polymerization step can be performed at ambient conditions, such as at room temperature, but it can be further accelerated by any mean adapted to induce and/or enhance polymerization, for instance by heating of the mixture. The pre-polymerization step can be performed in an inert atmosphere, such as under argon or nitrogen, in order to reduce or eliminate any environmental factors that could interfere with the pre-polymerization reaction (e.g., oxygen). The conditions for pre-polymerization, if performed, can depend on the type of T-PM, as well as on the selected cross-linker. In some embodiments, pre-polymerization can be performed at a temperature between 20° C. and 60° C., between 25° C. and 60° C., between 30° C. and 60° C., or between 40° C. and 60° C., or at higher temperatures, such as between 100° C. and 150° C. or between 150° C. and 200° C., and for at least 5 minutes, at least 10 minutes, at least 20 minutes, at least 30 minutes, at least 40 minutes, at least 50 minutes, at least 60 minutes, at least 120 minutes or at least 180 minutes. Typically, the duration of pre-polymerization does not exceed 24 hours, 18 hours, or 12 hours, when performed at relatively mild temperature, but can be shortened if performed at relatively higher temperatures (e.g., between 150° C. and 200° C.) which may require less than 8 hours, less than 5 hours or less than 4 hours. Following pre-polymerization, additives can optionally be added to the pre-polymerized compartment, and/or an aqueous compartment can be combined therewith to form the hair styling composition.

The hair styling composition (e.g., oil-in-water emulsion) can be readily applied following its preparation or within a time period during which it remains suitably stable and potent. For instance, if an emulsion, the composition can be applied as long as the oil droplets are within their desired size range (e.g., of no more than a few micrometers, typically less than 10 μm), provided that the T-PMs have not fully polymerized in vitro. More generally, the hair styling composition can be used as long as a sufficient amount of T-PMs is available to at least partially penetrate the hair fiber, so as to polymerize therein. In some embodiments, the single-phase composition or the emulsion is applied to the hair fibers within at most 30 minutes from its dissolution or emulsification, or within at most 20 minutes, at most 10 minutes, or at most 5 minutes.

In some embodiments, prior to applying the hair styling composition, either as a single-phase composition or as an oil-in-water emulsion, the hair fibers may be pre-treated.

A common pre-treatment that may be performed prior to applying the hair styling composition is a cleaning pre-treatment, wherein any residual materials that may be present on the hair, such as hair products, dirt or grease, are removed to clean the hair fibers. This can be done by applying any suitable cleaning products, such as sodium lauryl sulfate, this washing being followed by the rinsing of the hair fibers with excess water.

Another pre-treatment, which may follow cleaning or be performed independently, is a drying pre-treatment, wherein rinsing water or residual moisture can be removed from the hair. This removal of water molecules from the hair fibers, typically achieved by heating of the hair, is believed to break hydrogen bonds that may have formed either on the cuticle scales' surface and/or within the hair shaft.

As used herein in the specification, unless clear from context or otherwise stated, the term “residual moisture”, with regards to the hair fibers, refers to water that is present either on the outer surface of the cuticle scales, between and/or below the scales (i.e., in the cortex or medulla), originating from the hair being exposed to humidity (e.g., to ambient humidity or as a result of hair wetting). Understandably, complete removal of residual moisture is very difficult to realize, as the hair is always exposed to ambient humidity which is rarely null. Nevertheless, low levels of residual moisture are achievable, or can be temporarily achieved by applying energy, mainly thermal (i.e., heat), to the hair. Heat sufficient to achieve minor levels of residual moisture can be applied to the hair by any conventional method, e.g., using a hair dryer or a flat or curling iron for enough time. Regardless of the method employed to reduce the amount of water molecules in the hair, such a step can alternatively be referred to as a drying treatment or step.

When considering hair having at least a wavy appearance, one can readily visually assess that enough hydrogen bonds are broken by a drying pre-treatment, as sufficient drying results in a transient relaxation of the waves, the hair fibers being eventually completely flattened at the end of such a step, if so desired. Alternatively, as is the case for straight hair, the duration of a drying pre-treatment can be arbitrarily set as a function of the drying device being used and the temperature it may apply to the hair fibers. For instance, flat or curling irons which may directly apply by heat conduction temperatures of about 200° C. to the hair can achieve sufficient breakage of hydrogen bonds within a few minutes, whereas conventional hair dryers which depending on the distance from the hair they are used may apply relatively lower temperatures by heat convection, could require relatively longer drying duration. Typically, drying the hair fibers can be performed by heating areas of the hair fibers up to a temperature of at least 40° C., at least 50° C., at least 70° C., at least 80° C., or at least 100° C. for no more than 5 seconds at a time, such drying treatment taking up to 5 minutes for hair swatches when the heating proceeds from one end of the swatch to the other.

In some embodiments, the residual moisture level following such a drying treatment (if performed) and/or prior to application of the present compositions is at most 5 wt. %, at most 4 wt. %, at most 3 wt. %, at most 2 wt. % or at most 1 wt. % by weight of the hair fibers. Such amount can be determined by standard methods, using, for instance, thermogravimetric analysis, or near infrared technologies, such as opto-thermal transient emission radiometry.

Alternatively, or additionally, the heating that may inter alia contribute to the cleavage of hydrogen bonds within the keratin polymer and/or within the materials of the hair styling composition having penetrated the hair fibers, is the one a) optionally applied during the application of the composition (e.g., the composition being heated prior to its application); b) optionally applied during the incubation of the composition on the hair fibers; and/or c) applied during the styling of the hair fibers following the application of the composition. Regardless of its effect on hydrogen bonds, if any, the heating promotes the diffusion rate of the monomers/oligomers and/or the curing of the polymer within the hair fibers.

A third possible pre-treatment, which may follow cleaning and/or drying, or be performed independently, involves the application of a pre-treating composition intended to remain on the hair fibers during the performance of the hair styling method. The hair pre-treating composition can protect the hair fibers during the application of the hair styling composition, in particular during the application of heat, it can facilitate the performance of steps of the present methods, and/or it may enhance the properties of the hair styling compositions.

The hair pre-treating compositions should not undermine the effects sought by the present compositions and methods, and for instance should not interfere with the cuticles opening, with the migration of the styling composition towards the hair surface, with the penetration of the styling composition into the hair shaft, with the polymerization of the T-PMs or any like effect.

Typically, the hair pre-treating composition consists of an oil which can be applied to the hair fibers so as to form an ultra-thin oily layer on the surface of the fibers prior to their treatment with the hair styling composition.

In some embodiments, the oil used for this pre-treatment step or hair pre-treating composition has a solubility in water of 5 wt. % or less, 4 wt. % or less, 3 wt. % or less, 2 wt. % or less, or 1 wt. % or less, by weight of the water, as measured at a temperature of 25° C.

Factors rendering a hair pre-treating composition suitable for the present methods share similarities with some of the properties already described for the sake of the hair styling compositions and will only be mentioned briefly.

First, the hair pre-treating composition, which can be referred to as a pre-treating oil, should properly wet the hair fibers. For that purpose, the pre-treating composition or the oil therein should have a surface tension that is lower than the hair fiber surface energy. In some embodiments, the pre-treating composition or the oil have a surface tension of 35 mN/m or less, 30 mN/m or less, or 25 mN/m or less.

Secondly, in order to remain on the hair fibers and not evaporate during the application of energy, if so desired, the hair pre-treating composition or the oil therein should be essentially non-volatile during the process. Accordingly, in some embodiments, the pre-treating composition has a vapor pressure of less than 40 Pa, less than 35 Pa, or less than 30 Pa. In other embodiments, the pre-treating composition has a vapor pressure of more than 0.1 Pa, more than 0.2 Pa, or more than 0.5 Pa. In some embodiments, the pre-treatment composition (e.g., a pre-treating oil) has a vapor pressure between 0.1 Pa and 40 Pa, between 0.2 Pa and 35 Pa, or between 0.5 Pa and 30 Pa. The vapor pressure of such pre-treatment composition is measured at a temperature of 25° C.

In order to facilitate the coating of the hair fibers with a desired hair pre-treating composition, electrostatic attraction between the two can be promoted, the hair pre-treating composition (e.g., the oil pre-treatment) preferably having a zeta potential (ζₒ) that is sufficiently different from the hair fibers zeta potential (ζₕ). The ζₒ of the pre-treating composition should also be sufficiently different from the zeta potential of the styling composition (ζ_{c}), in order to allow attraction inter alia of the T-PMs to the oil pre-treatment layer formed on the hair fibers. Accordingly, in some embodiments, the delta zeta potential between the pre-treating composition and the hair (Δζ_{|o-h|}) and the delta zeta potential between the styling and the pre-treating compositions (Δζ_{|c-o|}), are each independently at least 5 mV, at least 10 mV, at least 15 mV, at least 20 mV, at least 25 mV, at least 30 mV, or at least 40 mV, all in absolute values. In some embodiments, Δζ_{|o-h|} and Δζ_{|c-o|} absolute values are within a range of 5 to 80 mV, 10 to 80 mV, 10 to 70 mV, 10 to 60 mV, 15 to 80 mV, 15 to 70 mV, 15 to 60 mV, 20 to 80 mV, 20 to 70 mV, 20 to 60 mV, 25 to 80 mV, 25 to 70 mV, 25 to 60 mV, 30 to 80 mV, 30 to 70 mV, 30 to 60 mV, 35 to 80 mV, 35 to 70 mV, or 35 to 60 mV.

As it is desired that the materials penetrating the hair shaft in priority are those participating in or promoting the in situ polymerization of the T-PM(s), it can be beneficial to select the hair pre-treating composition, if present in the method, to be substantially unable to penetrate into the hair. Hence, in some embodiments, the pre-treatment oil has a hair-penetrating ability as measured by weight gain of up to 5 wt. %, up to 4 wt. %, up to 3 wt. %, up to 2 wt. %, or up to 1 wt. % by weight of the hair fibers. Regardless of their ability to penetrate, or not, into the hair fibers, hair pre-treating compositions must not adversely interfere with the sought activity of the hair styling composition (e.g., prevent its polymerization, as can be tested in vitro).

In some embodiments, the pre-treating composition can be selected to be incompatible with the hair styling composition from a miscibility standpoint. For example, a pre-treating oil can have a miscibility of 5 wt. % or less, 4 wt. % or less, 3 wt. % or less, 2 wt. % or less, or 1 wt. % or less, by weight of the hair styling composition, as measured at a temperature of 25° C.

In such a case, it is believed that such lack of miscibility between the two compositions prompt excess hydrophobic droplets of hair styling composition to bead on the hair surface previously coated with a thin oily layer. Thus, while the thin layer of a pre-treatment composition allows for the penetration of the components of the styling composition, its presence can facilitate removing the part of the hair styling composition that did not penetrate the hair fibers. Excess styling composition may be removed along with the layer of the pre-treatment oil e.g., by washing the hair or wiping off. In such a case, a pre-treatment oil, in addition or alternatively to any other benefit it may provide, may improve the appearance, the feel, and/or the combability of the hair fibers at an earlier stage as compared to hair fibers treated with a same hair styling composition in absence of the pre-treating oil.

Whether any one of the afore-described optional pre-treatment steps was previously performed or not, the hair styling composition (e.g., oil-in-water emulsion) is applied to the hair fibers, and maintained on the hair typically for a period of at least 5 minutes, allowing the cuticle scales to swell and open, and thus granting the T-PMs and the curing facilitators, if present, access into the hair shaft. To facilitate penetration into the hair cortex, the molecules participating in or facilitating the internal polymerization (e.g., T-PMs, curing facilitators, co-solvents) preferably have a molecular diameter of less than 2 nm, less than 1.8 nm or less than 1.6 nm. The Inventors posit that once within the shaft, the monomers can bond to at least part of the broken hydrogen bonds in the hair fibers, preventing them from re-forming in their prior native state upon exposure to water. The T-PMs may additionally, or alternatively, polymerize without being bonded to the previously broken hydrogen bonds. Regardless of the mechanism of action, polymers resulting from the curing of the monomers having impregnated the hair fibers are able to constrain the hair fibers in their new shape. It is believed that the cured composition of the invention prevents water (either ambient or applied during wetting) from accessing the hair or diminishes its access, thereby reducing or delaying the ability of hydrogen bonds to form again, deferring the ability of the hair to revert to its native shape. Hence, while for simplicity the method is described in terms of breakage of hydrogen bonds and subsequent blockage of the broken bonds by attachment to T-PMs or other ingredients that may thereafter polymerize, this is not meant to rule out any additional rationale underlying the observed styling effect.

Sufficient time is provided for the monomers to impregnate the hair fibers and ensure their bonding, e.g., to at least part of the broken hydrogen bonds in the hair fibers. In some embodiments, the composition is allowed to remain in contact or is maintained applied on the hair fibers for a period of at least 10 minutes, at least 20 minutes, at least 30 minutes, at least 35 minutes, at least 40 minutes, at least 45 minutes, or at least 50 minutes. In some embodiments, the time period during which the composition remains applied on the hair fibers, alternatively referred to as the incubation time, is of at most 12 hours, at most 10 hours, at most 5 hours, at most 2 hours, or at most 1 hour. In particular embodiments, the composition is maintained on the hair fibers for a period of time between 5 minutes and 30 minutes, 10 minutes and 60 minutes, 30 minutes and 12 hours, between 30 minutes and 5 hours, between 40 minutes and 2 hours, or between 50 minutes and 2 hours. It is to be noted that conventional straightening methods may sometimes require longer period of times, some requiring 3-4 hours, or even 6-8 hours of application.

The composition can remain applied on the hair fibers at an ambient temperature (circa 23° C.), but this step can alternatively be performed at an elevated temperature of at least about 30° C., or at least about 40° C. In some embodiments, the temperature at which the composition can remain in contact with the hair fibers is of at most about 60° C., at most about 55° C. or at most about 50° C. In particular embodiments, the liquid composition is maintained on the hair fibers in a temperature range between 15° C. and 23° C., between 23° C. and 60° C., between 25° C. and 55° C., or between 25° C. and 50° C.

After said period of time, allowing for sufficient penetration of at least part of the T-PMs of the composition within the individual hair fibers, the monomers are subsequently at least partially cured, optionally in the presence of the curing facilitators, by application of energy, so as to effect at least partial polymerization.

Upon polymerization of the T-PMs, as can be more readily assessed within the liquid composition than within the hair fibers, the resulting polymer develops increasing glass transition temperature (Tg). In some embodiments, upon complete curing, the resulting T-PuP has a Tg of at least 50° C., at least 100° C., at least 150° C., or at least 200° C. Such Tg allows the polymerized T-PMs to remain intact under hot weather conditions, when washing the hair with hot water (around 45° C.), or even when being in an environment of elevated temperature, such as in a sauna (around 70° C.). As the synthetic polymer having formed within the hair fiber, thanks to its Tg, remains unaffected by such conditions or treatments, so is the modified shape of the hair achieved using the compositions and methods according to the present teachings.

In some embodiments, the energy allowing for at least partial curing of the composition (hence styling of the hair fibers) is a thermal energy, applied at a temperature of at least about 80° C., at least about 100° C., at least about 120° C. or at least about 140° C. In some embodiments, the heating temperature is at most 220° C., or at most 200° C. In particular embodiments, the temperature applied to achieve at least partial curing is in a range between 80° C. and 220° C., between 100° C. and 220° C., between 120° C. and 220° C., or between 140° C. and 200° C. It should be appreciated that the temperature provided by a heating device in order to at least partially cure the monomers is generally higher than the temperature perceived by the hair fibers. While given a long enough residence time (period during which the hair segment is exposed to the heat), the temperature of the hair fiber could eventually reach the temperature of heating, this is not generally the case and the temperature of the hair fibers at which curing may take place is typically of at least about 45° C., at least about 50° C., at least about 55° C., or at least about 60° C. In order to prevent irreversible damage to the hair fibers, the temperature of the hair fibers during the at least partial curing step is desirably of no more than 180° C., no more than 140° C., or no more than 100° C. The at least partial curing can be effected while styling the hair into the desired shape, e.g., by a hair dryer, or a flat or curling iron, so as to modify the native shape. This step, during which the hair fibers are mechanically constrained in a dynamic or static way to modify their shape (e.g., being pulled over a comb or brush, rolled on a roller, or contacted by a styling iron), can therefore alternatively be referred to as the styling step.

The time needed to reach at least partial curing at such temperatures is generally brief. Typically, an area of individual hair fibers perceiving a temperature of 100° C. or more may locally provide the partial polymerization of T-PMs therein within a few seconds, whereas hair fibers reaching a lower temperature of about 50° C. may require up to a few minutes (e.g., five minutes). The duration of time hair should be subjected to heating, hence should be perceiving a particular temperature adapted for curing, may depend on the shape of the hair to be modified and the new shape to be formed. A relatively mild modification may require less time than a relatively more dramatic change of shape.

A duration of time during which hair fibers should be at a suitable temperature can be independently tested in vitro by subjecting the oil phase of the composition due to be dissolved or emulsified to a temperature intended for the hair treatment, measuring the time it takes for the liquid phase to start solidifying (i.e., curing). When considering a mammalian subject, the amount of time allocated for the partial curing step (in other words, for the styling of the hair per se) would depend inter alia on the type of hair, its density on the scalp and its length, as well as on the device used to deliver the heat and its degree. Hence, on the level of an entire hair scalp, partial curing may take a few minutes, but generally no more than an hour. Such considerations apply to any other treatment of the hair fibers, the duration of time provided herein generally referring to periods suitable to any amount of hair fibers that can be simultaneously treated. If an entire hair scalp is to be treated step-wisely by repeating a same treatment for different batches of hair fibers, then the duration of treatment for the entire scalp may amount to the sum of durations due for the actual number of individual repeats of simultaneous treatments. For illustration, if five minutes are required to simultaneously treat a first batch of hair fibers, and an entire hair scalp is constituted of four such batches, then the treatment will be completed within about 20 minutes.

Prior to the at least partial curing, excesses of the hair styling composition (and of a pre-treating composition if any) are optionally removed from the outer surface of the hair fibers by rinsing the fibers with a rinsing liquid, so as to prevent formation of a thick coating on the surface of the hair fibers, and thus avoiding a tacky and coarse feel to the hair. The removal of such excesses can be further facilitated by the application of a suitable pre-treatment composition, such as an oil pre-treatment, as previously described. Rinsed fibers may also display improved heat transfer, accelerating partial curing therein.

Alternatively, or additionally, following the application of the hair styling composition and its incubation on the hair fibers, and optionally following rinsing, but prior to hair styling, a second composition consisting of curing facilitators can be applied to the hair fibers impregnated with the T-PMs. The composition that may be used in this optional step can be referred to as a curing composition. It may contain the same curing facilitators, selected from cross-linkers and curing accelerators previously described for the hair styling composition, and typically the curing composition consists of curing accelerators. In contrast with the hair styling composition, the curing facilitators (e.g., the curing accelerators) can be present in the curing composition in excess amount (e.g., at 5 wt. %) allowing the application of the curing composition to the hair fibers to be relatively brief (e.g., between 5 and 15 minutes, or less). The curing composition may additionally serve to rinse the fibers in addition to or instead of a rinsing solution.

Following the at least partial curing of the monomers, sufficient to achieve the desired modified shape, the hair fibers may optionally undergo further curing by application of further energy, preferably heat, to ensure additional curing of the composition. The further energy can be applied by the use of the above-mentioned styling instruments, e.g., hair dryer, or styling iron. In some embodiments, the further curing can be performed at temperatures as described for the at least partial curing of the third step, typically for a duration of time significantly longer than for partial curing. For instance, if hair fibers treated with a composition enabling at least partial curing with a specific styling device at a predetermined temperature within 20 minutes (as established by the fibers of the entire scalp displaying the desired modified shape), then an optional additional heating step favoring further curing would be performed for at least 40 minutes at least under the same conditions. Whereas partial curing is achieved while modifying the shape of the fibers, the step referred to herein as further curing is applied once the hair fibers are in the desired modified shape, so that concurrent mechanical constraining of the fibers to adopt the desired shape is no longer necessary. While further curing is expected to increase the extent of polymerization of the T-PMs within the hair fibers, it is not anticipated to achieve full curing (e.g., following which, polymerization can no longer take place).

In some embodiments, after heat curing (e.g., achieved during the styling step and the optional further curing), the hair fibers can be maintained, unwashed, to reduce exposure to water, allowing curing to further proceed, if applicable. The period during which washing of the hair fibers can be avoided may depend on the type of hair, the composition applied thereto, the procedure used to modify the native shape, the temperature, the relative humidity, the desired modified shape and the desired duration of said modification. Typically, assuming the hair fibers are maintained at room temperature at a relative humidity of about 40-60 RH %, washing of the hair may take place at least 18 hours after the termination of the at least partial curing (e.g., styling including mechanical constraint) or optional further curing step (e.g., heating without mechanical constraint). In some cases, washing can be deferred for at least 24 hours, for at least 36 hours, or for at least 48 hours. Usually, washing of hair styled according to the present method takes place within at most a week from styling. Hair styled according to the invention can be washed with any shampoo, not being restricted to the use of a particular one to avoid ruining the styling effect, as often necessary for conventional methods. Nevertheless, regular shampoos can be improved by including curing facilitators.

Advantageously, hair treated by the present hair styling compositions and the according methods is not only relieved of ongoing particular care, but the present teachings can also be suitable for hair fibers that have previously undergone other hair treatments (e.g., bleaching, coloring, styling, etc.). Such conventional treatments generally damage the hair, inflicting structural changes, e.g., physical and/or chemical, that might hamper subsequent hair treatment, such as styling by traditional methods (e.g., organic or Japanese). For instance, bleached hair might not be effectively straightened by the Japanese method due to the bleaching chemicals affecting hair components necessary for this method. In contrast, the compositions of the present invention are able to effectively style hair fibers regardless of any previous hair treatment they might have undergone.

FIG. 1 schematically depicts a FIB-SEM image of a clean hair fiber, wherein the cuticle scales 11 (being illustrated by sparsely dotted areas) are layered one on top of the other, separated by dark lines possibly indicative of cuticle-cuticle cell membrane complex (CMC).

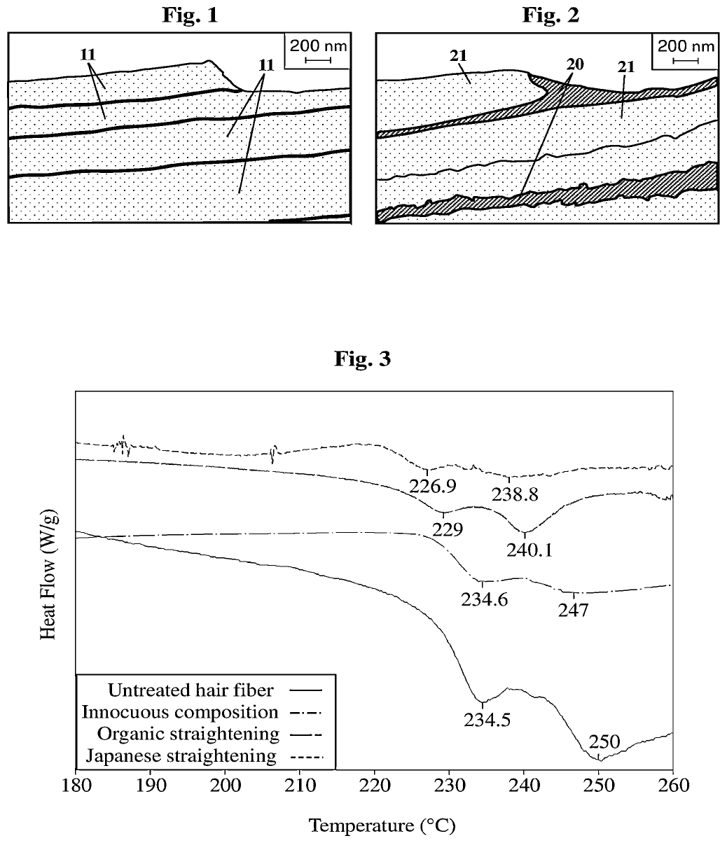

In comparison, FIG. 2 schematically illustrates a prophetic FIB-SEM image of a hair treated with a hair styling composition of the present invention, following curing. In the image, the cured composition 20 (marked by the dashed areas) is clearly visible located between the cuticle scales 21 of a treated hair fiber (marked by the sparsely dotted areas).

The methods of the present invention provide for durable hair styling, which keeps the hair fibers in the desired shape even after the hair is exposed to moisture—whether to water originating from the atmosphere humidity or following wetting or washing of the hair. The hair styling can be maintained for long periods of time, wherein the styled shape is not affected in a significantly detectable manner even after 5 shampoo washes or more. As shall be demonstrated with the working examples, in some embodiments the hair styling composition and method according to the present teachings provide long lasting modification of the hair shape, as evidenced by the ability of the treated hair to withstand 10 or more shampoo washes, 20 or more shampoo washes, 30 or more shampoo washes, 40 or more shampoo washes, or 50 or more shampoo washes.

While it cannot be ruled out that part of this “wash resistance” results from residual disseminated coating on the fibers' outer surfaces, the Inventors posit that as such an external coating tends to wear out relatively rapidly with washes, and the ability to style hair according to the present teachings can be attributed predominantly to the internal polymerization of the T-PMs. It is to be noted that this transient scattered coating is relatively thin, usually not exceeding an initial thickness of 1 μm, often being less than 0.5 μm thick, which in itself distinguishes hair fibers treated according to the present teachings from conventional styling methods relying on continuous external coatings of a few microns to constrain the fibers in a desirable shape. Without wishing to be bound by theory, it is believed that this transient thin coating of the hair fibers may temporarily protect the inner shaft so that the monomers having penetrated therein can further their curing, strengthening their polymerization, thus extending the hair styling durability.

As used herein, a composition providing for a modified shape able to resist 5 to 9 shampoo washes can be referred to as having a short term styling effect. A composition providing for a wash resistance of 10-49 shampoo cycles is said to provide for a semi-permanent styling, whereas compositions providing wash resistance to more than 50 shampoos can be said to provide permanent styling.

The rapid absence of a continuous external coat (insignificant for the present long lasting styling effects) is deemed advantageous, as methods relying on such peripheral constricting structures to durably maintain a straightened hair shape have often been found detrimental to hair health and natural look.

While the present compositions and methods are particularly beneficial for long lasting hair styling, for which the alternatives are typically deleterious to the hair and often to the health, they may additionally or alternatively be used for short term hair styling, the hair fibers regaining their native original shape following 2 to 4 shampoo washes.

FIG. 3 depicts the results of a DSC study showing that traditional hair straightening methods are damaging to the hair and illustrating the impact expected from an innocuous hair styling method, as anticipated for the hair styling compositions of the present invention.

As can be seen in the figure, the curve of a sample of hair fibers as would appear if treated with a composition of the present invention, is comparable to the curve of untreated, native hair sample, indicating no significant structural changes, hence damage to the hair. In contrast, the DSC curves of commercial hair straightening methods (organic and Japanese) show substantial changes from the native hair sample curve, indicating structural changes, which are to be expected when using such drastic hair styling methods. The DSC study is further detailed in Example 4 below.

Advantageously, hair fibers treated by the compositions according to the present teachings are expected to display at least one endotherm temperature within 4° C., within 3° C., within 2° C., or within 1° C. from similar untreated fibers, as measured by thermal analysis.

The non-damaging effect of the present compositions to hair fibers treated therewith can be confirmed or alternatively established by tensile testing, wherein various mechanical parameters can be compared between treated and similar untreated hair fibers, as described in Example 5 below. While fibers styled using conventional organic straightening are expected to show inferior mechanical properties compared to untreated fibers, fibers treated according to the present invention may display behavior similar or even superior to untreated fibers of similar nature. Without wishing to be bound by any particular theory, such improved properties, or at least absence of significant deterioration, are believed to stem from the presence of a polymerized version of the T-PMs within the inner parts of the hair fibers.

One mechanical parameter, where hair fibers treated by the present invention are expected to be at least as good as untreated hairs relates to the pressure (or force per cross-sectional area) required to break the hair, or break stress, measured at the break point in a strain-stress curve. A second mechanical parameter is hair toughness, which estimates the amount of energy the hair can absorb before breaking (i.e., the area under a strain-stress curve). Elastic modulus is another mechanical parameter, indicating the hair fibers' resistance to elastic deformation, where fibers treated by the present methods are expected to be at least comparable to untreated hair.

In some embodiments, the hair fibers treated by the compositions according to the present teachings, when measured by tensile strength analysis, display at least one of:
i) a break stress of at least 5%, at least 10%, at least 20% or at least 25% greater than the break stress of similar untreated fibers; and
ii) a toughness of 95% or more, 100% or more, 105% or more, 110% or more, 115% or more, or 120% or more of similar untreated hair fibers.

The methods of the present invention are suitable for any desired hair style and shape, such as straightening, curling, or rendering an intermediate shape, wherein the hair is relaxed to a form less wavy than its natural unmodified shape.

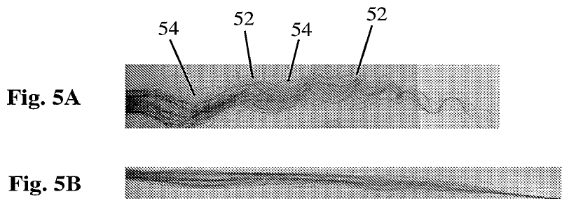

FIG. 5A shows an image of a natural, untreated curly black hair tuft, in which twists (e.g., peaks 52 and dips 54) in the hair fibers are clearly detectable. FIG. 5B, in comparison, shows an image of a sample of curly black hair, as would appear if treated with a hair styling composition described in the present invention, and straightened with a flat iron, the hair fibers showing a drastic decrease in the number of twists as compared to the untreated reference.

Advantageously, the present compositions allow restyling without necessitating application of a new composition. Hence, following a single pass of the method, embodiments of which have been described above, the method serving to modify the shape of the hair fibers from a native shape to a first modified shape, the hair fibers can be reshaped to a second modified shape. This can be achieved by bringing the hair fibers to a temperature above the Tg or softening temperature of the polymer formed during the first shaping, hence affording what may be referred to as “at least partial softening”. During and/or following such a step of at least partial softening, the hair fibers are formed in a desired second shape. The polymer is then allowed to regain a constraining structure adapted to retain the second shape, by allowing the temperature to decrease below its Tg or softening temperature while the hairs are maintained in the desired shape. The temperature can alternatively be actively lowered, for instance by blowing cool air on the hair. The second modified shape can be the same or different than the first modified shape. While this innovative restyling method has been described with respect to the softening of the polymer having previously penetrated within the fibers, it is believed that the heating applied to achieve such softening may additionally serve to decrease the water content. As previously explained, the elimination of residual water may, in turn, affect hydrogen bonding, enhancing the effect of the polymer having reformed upon cessation of its softening.

Advantageously, the present compositions allow “de-styling” when desired, by which it is meant that the hair fibers treated according to the present invention can regain their original shape without waiting for the effect of styling to vanish with time or for the regrowth of naturally shaped hair fibers. This can be achieved by subjecting the previously styled hair fibers to a temperature above the Tg or softening temperature of the polymer in the presence of water for a sufficient amount of time for the temperature to soften the polymer, and the water to penetrate the fibers. Without wishing to be bound by theory, it is believed that that such de-styling treatment could result in the softening of the polymer, thus possibly allowing a certain degree of cleavage of bonds that the polymer may have formed with moieties of the hair fibers prone to form hydrogen bonding. The presence of water during the de-styling treatment enables penetration of such molecules into the hair, resulting in the reformation of at least part of the hydrogen bonds naturally occurring in the untreated hair. Depending on the extent of reformation of the original hydrogen bonds of the hair fibers, and the form the polymer may adopt upon cooling back to a lower temperature no longer supporting its softening, the de-styling can be partial or complete, the hair accordingly returning less or more closely to its original shape. The de-styling process is believed to only affect the shape of the polymers remaining within the hair shaft, therefore, following de-styling, the hair fibers can, if desired, undergo an additional styling treatment, as previously described for restyling.

The Tg or softening temperature of the synthetic polymer within the hair fibers can be empirically assessed, for example in vitro. A sample of the hair to be restyled or de-styled can be collected from the hair scalp to be treated by such methods and placed in the intended re-/de-styling liquid (e.g., water). At this stage, the hair fibers of the sample have a particular modified shape. Temperature can be gradually raised and the ability of such temperature to relax the shape monitored. A temperature is deemed suitable for the at least partial softening of the polymer when the hair fibers lose their modified shape and revert towards their native shape. A suitable temperature may also depend on the duration of the sample incubation. Alternatively, the Tg of a thermally-curable polyol polymer (T-PP) formed by polymerizing in vitro the thermally-curable polyol monomers (T-PM) according to the method previously described can be determined by standard thermal analysis methods, e.g., DSC, such as described in ASTM E1356. In some embodiments, the Tg or softening temperature of the polymer is at least 40° C., at least 50° C., or at least 60° C., such softening temperature generally not exceeding 80° C. The duration of time the hair fibers should be subjected to such temperatures to achieve restyling or de-styling can be similarly determined. Typically, such treatments last at least 5 minutes, at least 10 minutes, at least 20 minutes, at least 30 minutes, at least 40 minutes, at least 50 minutes, or at least 60 minutes, generally not exceeding 4 hours or 3 hours, relatively higher temperatures requiring relatively shorter softening times. The hair styling compositions can be sold with guidance concerning the temperature and time needed to effect restyling or de-styling if desired.

Advantageously, the present compositions and methods are suitable for the styling of growing hair. The synthetic polymer formed by a first application of the hair styling composition is expected to be located in the segments of the hair fibers available above scalp at the time of application of the monomers. With time and hair growth, such segments are to be found more and more distal from the scalp, while the newly grown hair segments adjacent to the scalp would be devoid of such inner styling skeleton. It is believed that hair styling compositions applied at a later time following such hair growth would probably act mainly on the newly grown segments, the earlier treated segments being already “occupied” by previously formed synthetic polymer. However, since as explained the existing polymer can permit restyling or de-styling of the fibers, it may functionally merge with a polymer that would be newly formed in the new segments, providing a “styling continuity” along the entire fiber, preexisting and newly grown.

The present invention further provides a liquid composition for styling mammalian hair fibers, wherein the liquid composition is a single-phase composition comprising:

at least one T-PM, as herein described;

at least one curing facilitator selected from a cross-linker and a curing accelerator;

water; and

one or more co-solvents;

the liquid composition having a pH adapted to facilitate the penetration of the monomer within the hair fibers.

The present invention further provides a liquid composition for styling mammalian hair fibers, wherein the liquid composition is a curable oil-in-water emulsion comprising:

an oil phase containing at least one T-PM, as herein described and at least one curing facilitator selected from a cross-linker and a curing accelerator; and

an aqueous phase containing water at a pH adapted to facilitate the penetration of the monomer within the hair fibers;

each of the oil phase and the aqueous phase optionally further comprising one or more co-solvents;

the oil phase being dispersed within the aqueous phase and the oil-in-water emulsion having a pH adapted to facilitate the penetration of the monomer within the hair fibers.

In some embodiments, the liquid hair styling composition (e.g., oil-in-water emulsion) optionally further contains at least one additive, selected from a group comprising an emulsifier, a wetting agent, a thickening agent, and a charge modifying agent, as described above and further detailed herein.

Advantageously, the hair styling compositions according to the present teachings are devoid of known carcinogenic compounds. For instance, in some embodiments, the hair styling composition contains permissible trace amounts of such compounds, which depending on jurisdiction can be less than 0.5 wt. % formaldehyde, less than 0.2 wt. % formaldehyde, less than 0.1 wt. % formaldehyde, or even below permissible regulatory levels of less than 0.05 wt. % formaldehyde, less than 0.01 wt. % formaldehyde, less than 0.005 wt. % formaldehyde, less than 0.001 wt. % formaldehyde, or no formaldehyde, by weight of the composition. The same limited concentrations apply to products that may produce or act as formaldehyde (e.g., glyoxylic acid and its derivatives, or any other formaldehyde-releaser), to glutaraldehyde and to products that may produce or act as glutaraldehyde (e.g., 2-alkoxy-3,4-dihydropyran). These deleterious compounds, including their respective precursors or substituted forms (also termed formaldehyde-producing compounds or -releasers), such as Quaternium-15 (including for instance Dowicil™ 200; Dowicil™ 75; Dowicil™ 100; Dowco™ 184; Dowicide™ Q produced by Dow Chemical Company); imidazolidinyl urea (such as Germall™ 115 Ashland); diazolidinyl urea (such as Germall™ II); bromonitropropane diol (Bronopol); polyoxymethylene urea; 1,2-dimethylol-5,6-dimethyl (DMDM) hydantoin (traded as Glydant); tris(hydroxymethyl) nitromethane (Tris Nitro); tris(N-hydroxyethyl) hexahydrotriazine (Grotan® BK); and sodium hydroxymethylglycinate), can be referred to herein, individually and collectively, as small reactive aldehyde(s) (SRA(s)).

As appreciated by persons skilled in organic chemistry, SRA molecules need not be aldehyde per se and can be of additional chemical families as long as being able to form (e.g., by hydrolysis, degradation, reaction, and the like) deleterious aldehydes including formaldehyde and glutaraldehyde. Such formation can be triggered by conditions often encountered in hair styling, such as upon application of heat. Some of such precursors can entirely convert into formaldehyde or glutaraldehyde, one molecule of SRA yielding, optionally via intermediate products, one or more molecules of formaldehyde under ideal conditions, which may be extreme, whereas other precursors may convert only in part. Heximinium salts are one example of the latter.

In any event, assuming the SRA compounds are other than formaldehyde or glutaraldehyde, their weight amount in the composition would exceed the final weight amount of formaldehyde or glutaraldehyde that can be formed thereby. In particular embodiments, the hair styling composition contains less than 0.5 wt. % SRA, less than 0.2 wt. % SRA, less than 0.1 wt. % SRA, less than 0.05 wt. % SRA, less than 0.01 wt. % SRA, less than 0.005 wt. % SRA, less than 0.001 wt. % SRA, or no SRA, by weight of the composition. As can be appreciated, the hair styling composition will be deemed to be essentially free of SRA molecules if containing or producing during the hair styling method (e.g., upon heating of the composition) undetectable levels of formaldehyde.

As formaldehyde reacts with hair proteins, its substantial absence from the present hair styling compositions results in a corresponding absence of its reaction products in the treated hair fibers. Reaction products of formaldehyde depend on the amino acid it is reacting with, and, by way of example, reaction with cysteine yields thiazolidine and hemithioacetal, reaction with homocysteine yields thiazinane and hemithioacetal, reaction with threonin yields oxozolidine, and reaction with homoserine yields 1,3-oxazinane. Such reaction products can be detected in hair fibers by standard methods, including by nuclear magnetic resonance (NMR).

Thus, mammalian hair fibers styled according to the present methods, or with the present compositions, can be characterized by containing less than 0.2 wt. %, less than 0.1 wt. %, less than 0.05 wt. %, less than 0.01 wt. %, less than 0.005 wt. %, less than 0.001 wt. %, or being significantly devoid of reaction products between formaldehyde and amino acids. In some embodiments, the mammalian hair fibers treated according to the present teachings contain undetectable levels of at least one of thiazolidine, hemithioacetal, thiazinane, oxozolidine, and 1,3-oxazinane, as can be measured by NMR. As cysteine may account for up to 18% of the amino acid repeats of normal human keratin protein, the absence of thiazolidine and/or hemithioacetal in the hair fibers might be the most significant marker(s) for the corresponding absence of formaldehyde and formaldehyde forming products in the composition previously used to treat the hair.

In some embodiments, the hair styling composition is substantially devoid of amino acids, peptides and/or proteins. Proteins absent from the present compositions can be naturally occurring proteins, such as keratin and collagen, or synthetic and/or modified (e.g., hydrolyzed) forms thereof, and the lacking peptides may be smaller fragments of such proteins. For simplicity, such peptides may be named according to the larger protein they may be part of, and for instance can be referred to as keratin-related peptides or collagen-related peptides, when considering the proteins most frequently used in hair treatment.

The hair styling compositions according to the present invention are substantially devoid of such substances, if amino acids, peptides or proteins, and in particular keratin, collagen and their related peptides, constitute no more than 1 wt. % of the composition, their respective concentration being preferably of no more than 0.5 wt. %, of no more than 0.1 wt. %, or of no more than 0.05 wt. % by weight of the hair styling composition. In some embodiments, such substances are substantially absent (e.g., at about 0 wt. %) from the composition, accordingly. The presence or absence of such biomolecules can be determined by standard methods, for example by matrix-assisted laser desorption/ionization (MALDI) and related techniques, including for instance with a time-of-flight mass spectrometer (MALDI-TOF).

Thus, mammalian hair fibers styled according to the present methods, or with the present compositions, can be additionally or alternatively characterized by being significantly devoid of peptides and proteins, other than naturally formed ones. If the hair fibers were treated by a conventional method using naturally occurring proteins or related peptide fragments thereof, then hair fibers styled according to the present methods can in contrast be characterized by being significantly devoid of peptides of proteins naturally occurring in the hair fibers.

Identification of a hair styling compositions of the present invention can be performed by detecting functional groups characteristic of the essential components of the composition as described herein. Similarly, the use of such compositions according to the present methods can be deduced from the presence of such characteristic groups in extracts from hair styled fibers, as can be measured by standard methods and routine experimentation by persons skilled in analytical chemistry. For illustration, functional groups, such as isocyanates or —OH groups, can be detected by any suitable method known in the art, such as Fourier Transform Infrared Spectroscopy (FTIR).

In summary, mammalian hair fibers comprising in their inner part at least partially cured T-PMs of the present invention, forming a synthetic polymer within the fiber, can be characterized by at least one of the following features:
i) having less than 0.2 wt. % of a reaction product of formaldehyde and amino acids, the reaction product being selected from a group comprising thiazolidine, hemithioacetal, thiazinane, oxozolidine, and 1,3-oxazinane thiazolidine, by weight of the hair fibers;
ii) displaying at least one endotherm temperature within 4° C., within 3° C., within 2° C., or within 1° C. from untreated hair fibers, as measured by thermal analysis such as DSC;
iii) having a break stress of at least 5%, at least 10%, at least 20% or at least 25% greater than the break stress of similar untreated fibers, as measured by tensile analysis;
iv) having a toughness of 95% or more, 100% or more, 105% or more, 110% or more, 115% or more, or 120% or more of similar untreated hair fibers, as measured by tensile analysis; and
v) having less than 0.2 wt. %, less than 0.1 wt. %, less than 0.05 wt. %, less than 0.01 wt. %, less than 0.005 wt. %, or less than 0.001 wt. % of small reactive aldehydes (SRA) selected from: formaldehyde, formaldehyde-forming chemicals, glutaraldehyde, and glutaraldehyde-forming chemicals, by weight of the hair fibers.

In one embodiment, the mammalian hair fibers fulfill at least feature i) as above listed. In one embodiment, the mammalian hair fibers fulfill at least feature ii) as above listed. In one embodiment, the mammalian hair fibers fulfill at least feature iii) as above listed. In one embodiment, the mammalian hair fibers fulfill at least feature iv) as above listed.

In one embodiment, the mammalian hair fibers fulfill at least features i) and ii) as above listed. In one embodiment, the mammalian hair fibers fulfill at least features i) and iii) as above listed. In one embodiment, the mammalian hair fibers fulfill at least features i) and iv) as above listed. In one embodiment, the mammalian hair fibers fulfill at least the features i) and v) as above listed. In one embodiment, the mammalian hair fibers fulfill at least features iii) and iv) as above listed. In one embodiment, the mammalian hair fibers fulfill at least the features i), iii) and iv) as above listed. In one embodiment, the mammalian hair fibers fulfill at least the features i), ii), iii), and iv) as above listed. In one embodiment, the mammalian hair fibers fulfill at least the features i), ii), iii), iv) and v) as above listed.

The present invention also provides a kit for styling mammalian hair fibers, the kit comprising:
a) a first compartment containing at least one T-PM; and
b) a second compartment containing either: i. water at a pH adapted to facilitate the penetration of the monomer within the hair fibers, or ii. at least one pH modifying agent;
   wherein the kit further comprises at least one curing facilitator selected from: a cross-linker and a curing accelerator, wherein each of the at least one cross-linker and/or curing accelerator is independently placed in a same or different separate additional compartment(s); and
   wherein the mixing of said compartments' contents produces the hair styling composition (e.g., single-phase or oil-in-water emulsion) described above and further detailed herein.

In some embodiments, the at least one T-PM and curing facilitator are pre-polymerized prior to their placing in the kit.

In some embodiments, the components of the kit are packaged and kept in the various compartments under an inert environment, preferably under an inert gas, e.g., argon or nitrogen, and/or under any other suitable conditions preventing or reducing during the storage of the kit adverse reactions that may diminish efficacy of the composition. For instance, the kit should be stored at temperatures that would not induce polymerization, such as below 30° C., below 27° C. or below 25° C.

The kit may further comprise at least one curing facilitator, selected from a cross-linker and a curing accelerator. The curing facilitator (being a cross-linker or a curing accelerator) may be placed in the first or second compartment, depending on its reactivity with any one of the components of these compartments. For instance, a cross-linker that does not react with the T-PM at room temperature, can therefore be contained in the first compartment of a kit that is stored at such temperatures. Alternatively, if the curing facilitator tends to spontaneously react with any one of the components, it may be placed in a separate additional compartment.

The kit may optionally further contain at least one of a co-solvent, an emulsifier, a wetting agent, a thickening agent and a charge modifying agent, as previously detailed, which can be included in any one of the compartments described above, or in separate additional compartments. When considering the placement of such additives, oil-soluble components are preferably placed in compartments containing mostly oily components (e.g., the first compartment), and water-soluble components are preferably placed in compartments containing mostly aqueous components (e.g., the second compartment).

The kit typically includes a leaflet guiding the end-user on the manner of mixing the various compartments, the order of which may depend on the nature of the ingredients and/or the contents of the respective compartments. Generally, the proposed method of mixing and application shall enable the preparation of an effective and safe composition, to be applied within a time period suitable for its potency and intended use. For instance, if a third compartment containing a silane derivative as a curing facilitator is included in the kit, the leaflet may indicate first mixing of the curing facilitator with the T-PMs, then adding the contents of the aqueous compartment. Conversely, if a curing facilitator is present but is not a silane derivative, it may be included in the first compartment rendering the need for a separate third compartment superfluous.

In some embodiments, the ingredients of the various compartments are mixed, as may be instructed in such a leaflet, prior to the application of the final hair styling composition on the hair fibers. In such a case, the obtained composition may be used immediately, or maintained, un-applied, for up to 3 hours, up to 2.5 hours, up to 2 hours, up to 1.5 hours or up to 1 hour, prior to its application on the hair fibers.

Similarly, different timing and duration for application of the oil-in-water emulsion may conceivably be suggested depending on the desired duration of styling. For instance, if a short term styling is desired, the composition may be applied relatively later and/or for a shorter period of time than when a longer lasting styling is desired.

### EXAMPLES

### Materials

The materials used in the following examples are listed in Table 1 below. The reported properties were retrieved or estimated from the product data sheets provided by the respective suppliers. Unless otherwise stated, all materials were purchased at highest available purity level. N/A indicates that information is not available.

TABLE 1

| | | | | |
|---|---|---|---|---|
| Chemical Name | Product Name | MW | Supplier | CAS No. |

Thermally-curable Polyol Monomer (T-PM)

| | | | | |
|---|---|---|---|---|
| 2,3-bis[[(Z)-12- | Castor oil | 933.4 | N/A | 8001-79-4 |
| hydroxyoctadec-9- | | | | |
| enoyl]oxy]propyl (Z)-12- | | | | |
| hydroxyoctadec-9-enoate | | | | |

Cross-Linkers

| | | | | |
|---|---|---|---|---|
| 5-Isocyanato-1- | Isophorone | 222.28 | Sigma-Aldrich | 4098-71-9 |
| (isocyanatomethyl)-1,3,3- | diisocyanate | | | |
| trimethylcyclohexane | | | | |

pH-modifying agents

| | | | | |
|---|---|---|---|---|
| Ammonium hydroxide | NH₄OH | 35.05 | Sigma-Aldrich | 1336-21-6 |
| (25 wt. % aqueous solution) | | | | |

Additional agents

| | | | | |
|---|---|---|---|---|
| SLS | Sodium lauryl | 313.53 | Sigma-Aldrich | 110863-24-6 |
| | sulfate | | | |

### Equipment

### Vortex: Vortex-Genie 2 (by Scientific Industries, USA)

Flat iron: Babyliss® I-Pro 235 Intense protect

### Shaker: Digital Orbital Shaker TOU 50 (MRC Lab, Israel)

Oven: Heraeus oven, UT 12 (Thermo Scientific, USA)
Hair dryer: Itamar superturbo Parlux 4600 (Parlux®, Italy)
Differential Scanning calorimeter: DSC Q2000 (TA Instruments, USA)
Tensile tester: MTT157 (Dia-Stron, United-Kingdom)
Gas chromatographer GC-MS: GCD G1800A (HP, USA)

### Example 1: Preparation of Hair Styling Compositions Containing T-PM

### T-PM Mixture:

In a 20 ml vial, 0.9 g of castor oil (as T-PMs) were placed. 0.1 g of isophorone diisocyanate cross-linker were added, and the vial contents were mixed by vortex for about 30 seconds until a homogeneous mixture was obtained.

### Aqueous Mixture:

Alkaline water having a pH of 10 was prepared by combining 100 g of deionized water with 5 drops of ammonium hydroxide, amounting to about 0.075 g of the base.

### Oil-In-Water Emulsion:

Into a separate 20 ml vial, 0.4 g of the T-PM mixture were placed, and 15.8 g of the aqueous mixture were added and mixed together by hand for about 10 seconds, resulting in composition Pm1.

Similarly, composition Pm2 was prepared, wherein 0.2 g of castor oil and isophorone diisocyanate were mixed as described above, and the whole mixture was combined with the aqueous mixture to obtain the oil-in-water emulsion.

These two compositions are reported in Table 2, wherein the reported values correspond to the concentration of each ingredient in weight % (wt. %) by weight of the total emulsion.

TABLE 2

Composition

| | |
|---|---|
| Pm1 | Pm2 |

T-PMs compartment

| | | |
|---|---|---|
| Castor oil | 2.2 | 1.25 |
| Isophorone diisocyanate | 0.3 | 1.25 |

Aqueous compartment

| | | |
|---|---|---|
| Deionized water (pH 10) | 97.5 | 97.5 |

The hair styling compositions so prepared were stored at room temperature until further use, their application to hair samples being typically performed within 1 minute from the final mixing step. The pH, as well as the zeta potential, of the composition can be measured by standard methods using suitable instrumentation.

The presence of aldehydes, and specifically formaldehyde in the obtained compositions can be checked by gas chromatography-mass spectrometry (GC-MS), according to standard methods (e.g., NIOSH 2539 for aldehydes in general and NIOSH 2541 specifically for formaldehyde). Samples of the compositions can be heated in order to assess the formation of formaldehyde at various temperatures (e.g., heating to 220° C. to induce at least partial curing). The aldehydes and formaldehyde concentrations are expected to be less than 0.2 wt. %, and even below the level of detection, namely less than 1 ppm (i.e., less than 0.0001 wt. %). As readily appreciated, when using hair compositions substantially devoid of such SRA, hair fibers treated using the same are accordingly essentially free of such materials.

### Example 2: Hair Straightening Using Hair Styling Compositions

The ability of the present hair styling compositions to enable a durable change in shape of hair fibers was tested by straightening curly hair tufts.

The hair tufts used for testing the straightening ability of the present oil-in-water emulsions were black, and curly of Brazilian origin (approximately 40 cm long). Each tuft was glued together at one tip with epoxy glue, and weighted approximately 0.6-1.3 g, including the glued tip.

The curly hair tufts were all washed at 38-40° C. with tap water containing 5% sodium lauryl sulfate to remove any materials adhered to the hair (e.g., dirt or oils), and hanged to dry at room temperature for at least 1 hour, during which time the hair tufts regained their native shapes.

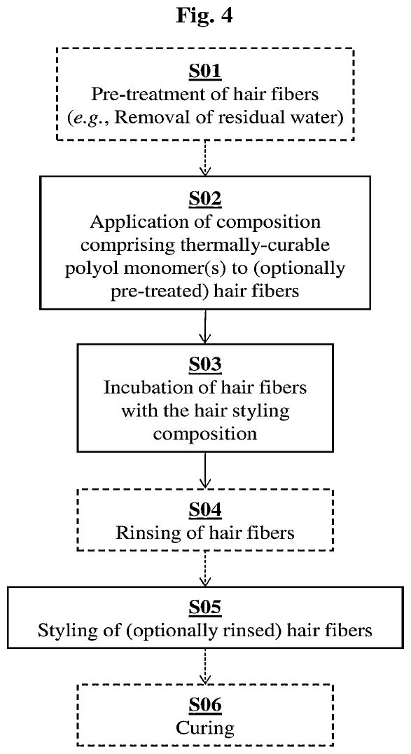

The basic treatment and straightening procedures which were applied to the clean hair samples are described below, and are schematically depicted in FIG. 4, which shows a simplified diagram of the different steps. While for simplicity, the compositions or methods can be referred to as “straightening”, in the present examples this term, which otherwise describes a particular hair styling effect of “complete flattening” of the hair fibers, is intended to encompass any significant shape modification, wherein the hair is relaxed to a form less wavy than native shape.

### Procedure:

1. (Optional) pre-treatment of hair fibers (as depicted in step S01 of FIG. 4): residual water can be removed from the dried clean hair tufts, e.g., by using a flat iron, passed 4 times over the tufts at a temperature of 200° C., resulting in the hair tufts being straightened.
2. Application of the composition (as depicted in step S02 of FIG. 4): the optionally heat-straightened hair tufts were dipped in a 100 ml plastic cup containing about 15-20 g of a hair styling composition, such as the oil-in-water emulsions prepared in Example 1.
3. Incubation of the composition (as depicted in step S03 of FIG. 4): the cups containing the hair tufts samples dipped in the various styling compositions were maintained for 30 minutes at room temperature (circa 23° C.).
4. Rinsing of the hair fibers (as depicted in step S04 of FIG. 4): the hair tufts so treated were thoroughly rinsed with tap water at a temperature of about 38-40° C. to eliminate excess composition in view of the method of experimental application, and then dried using a hair dryer for 2-3 minutes.
5. Styling of the hair fibers (as depicted in step S05 of FIG. 4): the rinsed treated hair-tufts were then straightened using a flat iron, at a temperature of 220° C. for 2-5 minutes (about 15-50 passes), depending on the tuft length, until the tufts were completely dried and in the desired modified shape. This step allows at least partial curing of the monomer(s).
6. (Optional) curing of the polymerizable styling composition (as depicted in step S06 of FIG. 4): the hair tufts, straightened and dried following step 5, can be exposed to further heating using a hair dryer or an oven, to ensure that the monomers having polymerized therein are further cured. When the T-PMs, T-POs, or T-PPs within the hair fibers are further cured using a hair dryer, the hair samples are maintained on a brush, and the hair dryer blowing air at a temperature of 150-220° C. is rapidly moved at a short distance over the tufts about 15 times, so that the hair fibers perceive an elevated temperature of at most 220° C. for a few seconds. When further curing is performed in an oven, the hair samples are maintained at 200° C. for 4 minutes, to reproduce the conditions of standard hair drying techniques.

It is to be noted that not all steps described in the present example, performed to illustrate the efficacy of the present compositions and methods in laboratory settings, as shall be supported in the following examples, are necessary in the conventional use of such compositions and methods (e.g., at home or in a hair salon). In the present example, while the composition can be applied on clean hair and/or on hair treated to remove residual water, such pre-treatment of hair fibers prior to the application of the composition is considered not essential (i.e., step S01 is optional, and its surrounding block in FIG. 4 was accordingly marked by a dashed contour), and was not performed. Similarly, while for the purpose of rapidly assessing efficacy, all hair samples were subjected to further curing (S06), in a routine use of hair styling compositions according to the present teachings, the process could end following the styling step (S05) upon sufficient drying having achieved the desired modified shape.

Conversely, additional steps may be used, or present steps modified. For instance, following optional rinsing (S04), a curing composition comprising excess amount of a curing facilitator may be briefly applied or the rinsing may be performed with a dedicated solution, other than tap water. Similarly, prior to styling of the hair fibers (S05), the hair can be treated with a formulation protecting the hair from damages that may result from the temperature applied during styling. Such a heat-protective formulation can contain or consist of oils having a relatively high smoking point at a temperature above the one applied for styling. Silicon oils can be used for this purpose.

### Example 3: Durability of the Hair Straightening

The hair tufts, treated with the compositions of the present invention as described in Example 2, were subjected to a series of washings 48 hours after the curing step 6. In each washing cycle, the hair tufts were massaged twice between the fingers of the operating person to ensure full coverage and intimate contact, from tip to tip, with a standard shampoo (Shea Natural Keratin Shampoo by Saryna Key, Israel) for about 30 seconds, rinsed with tap water at about 40° C., wiped and hung for at least 10 minutes to dry. The washing cycles were performed no more than twice a day, so as to mimic a plausible high frequency washing of a human subject.

The hair fibers treated with composition Pm1 sustained 5 washing cycles, and fibers treated with composition Pm2 sustained 10 washing cycles, which is indicative of the durability of the hair styling provided by the present compositions and method. This number can also be referred to as the “wash resistance” afforded by a particular composition under the conditions it was applied and tested.

### Example 4: Differential Scanning Calorimetry (DSC) Study

Keratin hair fibers demonstrate characteristic endothermic peaks in a number of thermal analytical methods, each peak being indicative of chemical changes occurring near the various temperatures. The hair samples treated according to Example 2 or 3 can be analyzed by DSC to assess the effect of the composition of Example 1 on the physico-chemical properties of the hair fibers and compare them to an untreated reference of a same hair type.

The reference and treated hair samples are cut into small pieces (about 2 mm long) using regular scissors. For each measurement, about 5 mg of hair pieces are placed in a 70 μl platinum DSC crucible. The crucible is kept open during measurements.

The samples are placed in a Differential Scanning calorimeter, and DSC measurements are carried out. Specifically, the samples are heated to 400° C. at a rate of 10° C./min under nitrogen, while data acquisition and storage are performed.

The stored data is plotted to obtain DSC curves for each of the hair samples and values of endotherm points are retrieved. If the modified and native hair fibers display at least one essentially similar endotherm temperature, the composition having achieved this modification is deemed innocuous. Endotherm temperatures of two materials or hair fibers can be considered essentially similar if within 4° C., 3° C., 2° C., or 1° C., from one another.

FIG. 3 depicts the results of a DSC study showing how a non-damaging hair styling method, such as proposed by the present invention, may keep the hair unharmed, as opposed to traditional methods. As can be seen in the figure, the curve of a sample of hair fibers treated with a hypothetical innocuous composition of the present invention would be comparable to the curve of untreated, native hair sample, indicating no significant structural changes. The solid line at the bottom of the plot depicts the curve of untreated curly black hair fibers. Two endotherms are observed, at 234.5° C. and 250° C., which are the characteristic temperatures for hair fibers. The first endotherm around 234.5° C. is believed to indicate the melting of α-keratin in the fiber, while the second endotherm around 250° C. is believed to indicate the keratin decomposition and breaking of the di-sulfide bonds.

In contrast, the DSC curves of commercial hair straightening methods (organic and Japanese) actually tested against the untreated reference show substantial changes from the native hair sample curve, indicating structural changes, which are to be expected when using such drastic hair styling methods.

Such measurements can alternatively be obtained from other methods of thermal analysis, such as by thermomechanical analysis (TMA) or dynamic mechanical analysis (DMA).

### Example 5: Mechanical Properties of Hair Fibers

The hair samples treated according to Example 2 or 3 can be analyzed by tensile testing to assess the effect of the compositions of the present invention (such as prepared in Example 1) on mechanical properties of the hair fibers and compare them to an untreated reference of a same hair type.

Ten hair fibers are taken from each one of a reference sample and a treated hair sample, and standardized by maintaining them under the same conditions for three days (e.g., a temperature of 25° C. and 45% RH). The hair fibers are then cut to a length of 30 mm, their cross-section is measured by confocal laser microscopy, taking into account both the largest radius and the smallest radius of typically elliptical hair fibers. The tensile strength parameters, break stress, toughness and elastic modulus, are measured for the examined hair fibers by tensile tester (at 100% extension limit, 20 mm/min extension rate, 2 g gauge force, 5 g break detection limit and 2000 g maximum force). The average results for the ten fibers of the treated hair sample are compared to those of the reference sample.

The break stress of the treated hair fibers is expected to be at least 5%, at least 10%, at least 20% or at least 25% greater than the break stress of similar untreated fibers. Furthermore, the treated hair fibers are expected to have a toughness of 95% or more, 100% or more, 105% or more, 110% or more, 115% or more, or 120% or more of similar untreated hair fibers. The elastic modulus of both treated and untreated samples is expected to be comparable.

### Example 6: Oil Pre-Treatment of Hair

While styling of hair fibers as described in Example 2 yielded satisfactory results (as evidenced by the afforded wash resistance described in Example 3), the procedure can be modified by including an oil pre-treatment step.

### Screening for Suitable Oils

Lack of Penetration:

The penetration of an oil candidate to the hair can be assessed as follows: a group of hair fibers, untreated by the compositions of the present invention, is weighed and placed in a cup containing the tested oil for a sufficient amount of time to allow possible penetration into the hair. After that time, the hair fibers are removed from the oil, wiped clean, and weighed again. Any increase in hair weight compared to their weight before their immersion in the oil can be attributed to the oil having penetrated into the fibers. Oils that cause a weight gain of less than 5% are considered suitable for their further screening as pre-treatment oils.

Lack of Polymerization Inhibition:

An inhibitory activity by an oil candidate can be assessed by applying a thin layer of the tested oil on a glass slide, followed by the application of a layer of a curable styling composition according to the present teachings which is to be tested for compatibility with the proposed pre-treating oil. The glass slide is then subjected to application of energy at appropriate temperature and for a sufficient amount of time to induce full curing of the styling composition (e.g., placing on a hot plate or in an oven). The slide with the cured layer of styling composition is allowed to cool. The cured layer is then peeled away from the slide and wiped clean of any residual oil that was applied beneath it on the slide. If the side of the cured composition that was in contact with the oil remains tacky, this is indicative that the curing was not complete, in which case the tested oil is believed to have an inhibitory effect on proper polymerization of the hair styling composition. Conversely, if the sides in former contact with the oil and with the air are similarly non-tacky, then the tested oil is considered suitable for further screening as a pre-treatment oil.

Lack of Miscibility:

The miscibility of an oil candidate with the styling composition to be applied thereon can be tested as follows: 0.05 g of the tested oil are added to 0.95 g of the hair styling composition under study, thoroughly mixed by vortex for 10 seconds and the mixture is allowed to separate into distinct phases. Oils that are found immiscible with the styling composition are deemed suitable as pre-treatment oils for the later application of said composition.

Pre-Treatment with the Selected Oils

Virgin hair fibers can be pre-treated with each one of the selected oils prior to the first step of the procedure described in Example 2 of applying the hair-styling composition.

Into a 100 ml plastic cup, 20 g IPA are mixed by hand with 0.2 gr of the pre-treatment oil. Hair tufts, previously washed with sodium lauryl sulfate, rinsed and dried as described in Example 2, are dipped in the oil/IPA mixture and maintained for 5 minutes at room temperature. The hair tufts are then rinsed with tap water and blow dried for a few minutes until completely dried.

The pre-treated tufts are styled with the hair styling composition prepared in Example 1, according to the procedure described in Example 2, and the durability of the styling treatment is tested as described in Example 3.

It is expected that such oil pre-treatments would allow the styling activity of the tested composition, while improving the feel and combability for the tested hair tufts, as can be assessed by trained operators.

Analysis by FIB-SEM microscopy can be further performed, in order to assess the effect of the oil pre-treatment on the transient coating that may initially form on the outer surface of treated hair fibers. It is expected that after a same number of washes, hair fibers pre-treated with the oil would not display a detectable transient coating on their outer surfaces compared to hair fibers which were not pre-treated with the oil.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

Although the present disclosure has been described with respect to various specific embodiments presented thereof for the sake of illustration only, such specifically disclosed embodiments should not be considered limiting. Many other alternatives, modifications and variations of such embodiments will occur to those skilled in the art based upon Applicant's disclosure herein. Accordingly, it is intended to embrace all such alternatives, modifications and variations and to be bound only by the spirit and scope of the disclosure and any change which come within their meaning and range of equivalency.

In the description and claims of the present disclosure, each of the verbs “comprise”, “include” and “have”, and conjugates thereof, are used to indicate that the object or objects of the verb are not necessarily a complete listing of features, members, steps, components, elements or parts of the subject or subjects of the verb. Yet, it is contemplated that the compositions of the present teachings also consist essentially of, or consist of, the recited components, and that the methods of the present teachings also consist essentially of, or consist of, the recited process steps.

As used herein, the singular form “a”, “an” and “the” include plural references and mean “at least one” or “one or more” unless the context clearly dictates otherwise. At least one of A and B is intended to mean either A or B, and may mean, in some embodiments, A and B.

Unless otherwise stated, the use of the expression “and/or” between the last two members of a list of options for selection indicates that a selection of one or more of the listed options is appropriate and may be made.

Unless otherwise stated, when the outer bounds of a range with respect to a feature of an embodiment of the present technology are noted in the disclosure, it should be understood that in the embodiment, the possible values of the feature may include the noted outer bounds as well as values in between the noted outer bounds.

As used herein, unless otherwise stated, adjectives such as “substantially”, “approximately” and “about” that modify a condition or relationship characteristic of a feature or features of an embodiment of the present technology, are to be understood to mean that the condition or characteristic is defined to within tolerances that are acceptable for operation of the embodiment for an application for which it is intended, or within variations expected from the measurement being performed and/or from the measuring instrument being used. When the term “about” and “approximately” precedes a numerical value, it is intended to indicate +/−15%, or +/−10%, or even only +/−5%, and in some instances the precise value. Furthermore, unless otherwise stated, the terms (e.g., numbers) used in this disclosure, even without such adjectives, should be construed as having tolerances which may depart from the precise meaning of the relevant term but would enable the invention or the relevant portion thereof to operate and function as described, and as understood by a person skilled in the art.

While this disclosure has been described in terms of certain embodiments and generally associated methods, alterations and permutations of the embodiments and methods will be apparent to those skilled in the art. The present disclosure is to be understood as not limited by the specific embodiments described herein.

Certain marks referenced herein may be common law or registered trademarks of third parties. Use of these marks is by way of example and shall not be construed as descriptive or limit the scope of this disclosure to material associated only with such marks.

We claim:

1. A method of styling mammalian hair fibers having a native shape, the method comprising:
a) applying to individual hair fibers a hair styling composition, the hair styling composition comprising at least one water-insoluble thermally-curable polyol monomer (T-PM) having an average molecular weight of 10,000 g/mol or less, at least one curing facilitator miscible therewith and water;
b) allowing the hair styling composition to remain in contact with the hair fibers for at least 5 minutes; and
c) applying thermal energy to at least partially cure at least part of the T-PMs within the hair fibers, said curing occurring while the hair fibers are at a temperature of at least 50° C., so as to obtain treated hair fibers;
wherein the hair styling composition contains less than 0.1 wt. % of small reactive aldehydes (SRA), the SRA being selected from formaldehyde, formaldehyde-forming chemicals, glutaraldehyde, and glutaraldehyde-forming chemicals.

2. The method as claimed in claim 1, wherein the thermal energy is applied while the hair fibers are in a desired modified shape, the modified shape being different from the native shape.

3. The method as claimed in claim 1, wherein the at least one water-insoluble thermally-curable polyol monomer (T-PM) is characterized by having at least two hydroxyl groups, and is selected from: polyalkylene glycols, glycerin, polyether polyols, amine-based polyols, polyester polyols and silanes containing at least two hydroxyl groups.

4. The method as claimed in claim 1, wherein the at least one curing facilitator is selected from a cross-linker and a curing accelerator, the curing facilitator being adapted to be in a same phase as the T-PM within the hair fibers.

5. The method as claimed in claim 1, wherein:
I. the hair styling composition further comprises at least one co-solvent, the at least one co-solvent being in an amount sufficient for the formation of an oil-in-water emulsion, the at least one T-PM being in an oil phase of the emulsion and at least one co-solvent being adapted to be in a same phase as the T-PM within the hair fibers; and/or
II. the hair styling composition further comprises at least one electromagnetic energy (EM)-curable monomer or the T-PM further including an EM-curable moiety, wherein further to the thermal energy applied in step c), EM energy is applied to effect at least partial curing of the at least one EM-curable monomer or moiety, the EM energy being applied prior to, concomitant with or subsequent to the thermal energy.

6. The method as claimed in claim 1, wherein prior to applying the hair styling composition to the hair fibers, A—the at least one T-PM and/or the at least one curing facilitator are pre-polymerized prior to mixing with the water, and/or B—the hair fibers are pre-treated by at least one of: a) cleaning the hair fibers; b) drying the hair fibers; and c) applying a pre-treating composition to the hair fibers.

7. The method as claimed in claim 6, wherein the pre-treating composition comprises an oil characterized by the following structural features:
i. the oil has a solubility in water of 5 wt. % or less, 4 wt. % or less, 3 wt. % or less, 2 wt. % or less, or 1 wt. % or less, by weight of the water, as measured at a temperature of 25° C.;
ii. the oil has a miscibility within the hair styling composition of 5 wt. % or less, 4 wt. % or less, 3 wt. % or less, 2 wt. % or less, or 1 wt. % or less, by weight of the hair styling composition, as measured at a temperature of 25° C.;
iii. the oil has a vapor pressure of less than 40 Pascal, less than 35 Pascal, or less than 30 Pascal, as measured at a temperature of 20° C.;
iv. the oil has a vapor pressure of more than 0.1 Pascal, more than 0.2 Pascal, or more than 0.5 Pascal, as measured at a temperature of 20° C.;
v. the oil has a surface tension lower than the surface energy of the hair fibers, said surface tension of the oil being optionally 35 mN/m or less, 30 mN/m or less, or 25 mN/m or less;
vi. the oil has a hair-penetrating ability of up to 5 wt. %, up to 4 wt. %, up to 3 wt. %, up to 2 wt. %, or up to 1 wt. % by weight of the hair fibers;
vii. the oil has a zeta potential ζₒ differing from a zeta potential of the composition ζ_{c}, in absolute terms, by at least 5 mV; and
viii. the oil is substantially devoid of an inhibitory activity with regards to curing of the T-PM(s).

8. The method as claimed in claim 1, further comprising following step b), at least one of I] removing excess hair styling composition from the hair fibers surface by rinsing the fibers with a rinsing liquid prior to applying energy to effect at least partial curing, the rinsing liquid optionally including at least one of a detergent and a curing facilitator, and II] applying to the hair fibers a curing composition comprising a curing facilitator; and/or further comprising following step c), at least one of III] washing the fibers with a washing liquid, and IV] conditioning the fibers with a conditioning liquid.

9. The method as claimed in claim 1, wherein the treated fibers and the untreated fibers display at least one endotherm temperature within 4° C., within 3° C., within 2° C., or within 1° C. from one another as measured by thermal analysis.

10. The method as claimed in claim 1, wherein the pH of the composition enables penetration of at least a part of the T-PM(s) into the hair fibers, said pH being in a range of 1 to 3.5 or 5 to 11.

11. A hair styling composition for modifying a shape of mammalian hair fibers, the composition comprising a) at least one water-insoluble thermally-curable polyol monomer (T-PM) having an average molecular weight of 10,000 g/mol or less; b) at least one curing facilitator miscible with at least one of the T-PM(s); and c) water; wherein the hair styling composition is further characterized by one of more of the following features:
a—the hair styling composition contains less than 0.1 wt. % of formaldehyde, formaldehyde-forming chemicals, glutaraldehyde, and glutaraldehyde-forming chemicals;
b—the hair styling composition contains less than 1 wt. % of amino acids;
c—the hair styling composition contains less than 1 wt. % of peptides; and
d—the hair styling composition contains less than 1 wt. % of proteins.

12. The hair styling composition as claimed in claim 11, wherein the at least one water-insoluble thermally-curable polyol monomer (T-PM) is characterized by having at least two hydroxyl groups, and is selected from: polyalkylene glycols, glycerin, polyether polyols, amine-based polyols, polyester polyols and silanes containing at least two hydroxyl groups.

13. The hair styling composition as claimed in claim 11, wherein a combined concentration of the at least one T-PM is at least 0.1 wt. %, at least 0.5 wt. %, or at least 0.9 wt. %, and at most 5 wt. %, at most 3 wt. %, or at most 2 wt. % by weight of the hair styling composition.

14. The hair styling composition as claimed in claim 11, wherein the at least one curing facilitator is selected from a cross-linker and a curing accelerator, the curing facilitator being adapted to be in a same phase as the T-PM within the hair fibers.

15. The hair styling composition as claimed in claim 11, wherein the at least one curing facilitator is at least one cross-linker, optionally selected from linear or branched, substituted or unsubstituted alkyl isocyanates, aromatic isocyanates, silane isocyanates and mixture thereof, wherein a combined concentration of the at least one cross-linker is further optionally at least 0.001 wt. %, at least 0.005 wt. %, at least 0.01 wt. %, at least 0.05 wt. %, at least 0.1 wt. %, or at least 0.5 wt. %, and at most 10 wt. %, at most 5 wt. %, at most 2.5 wt. %, at most 2 wt. %, or at most 1.5 wt. %, by weight of the hair styling composition.

16. The hair styling composition as claimed in claim 11, wherein the at least one curing facilitator is at least one curing accelerator, optionally selected from strong bases and amines, wherein a combined concentration of the at least one curing facilitator is further optionally at least 0.001 wt. % and at most 5 wt. % by weight of the hair styling composition.

17. The hair styling composition as claimed in claim 11, wherein the composition further comprises at least one A—co-solvent selected from the group consisting of: C₁-C₁₀ alcohols having at least one hydroxyl group, water-miscible ethers, aprotic solvents, and mineral or vegetal oils; the co-solvent being in an amount controlling a form of the composition, the form of the hair styling composition being an oil-in-water emulsion or a single-phase composition; and/or B—additive selected from a group comprising an emulsifier, a wetting agent, a thickening agent and a charge modifying agent.

18. The hair styling composition as claimed in claim 11, the composition having a pH in a range of 1 to 3.5 or 5 to 11.

19. Mammalian hair fibers comprising in an inner part thereof at least partially cured thermally-curable polyol monomers (T-PMs), forming a synthetic polymer having a softening temperature, wherein the hair fibers are characterized by at least one of:
i) having less than 0.2 wt. % of a reaction product of formaldehyde and amino acids, the reaction product being selected from a group comprising thiazolidine, hemithioacetal, thiazinane, oxozolidine, and 1,3-oxazinane thiazolidine;
ii) displaying at least one endotherm temperature within 4° C., within 3° C., within 2° C., or within 1° C. from untreated hair fibers, as measured by DSC;
iii) having a break stress of at least 5%, at least 10%, at least 20% or at least 25% greater than the break stress of similar untreated fibers; and
iv) having a toughness of 95% or more, 100% or more, 105% or more, 110% or more, 115% or more, or 120% or more of similar untreated hair fibers;
wherein the T-PMs correspond to at least one T-PM of a hair styling composition according to claim 11.

20. The mammalian hair fibers of claim 19, further characterized by having less than 0.1 wt. % of small reactive aldehydes (SRA) selected from: formaldehyde, formaldehyde-forming chemicals, glutaraldehyde, and glutaraldehyde-forming chemicals.
